(19) United States
(12) Patent Application Publication
(10) Pub. No.: US 2024/0226128 A1
(43) Pub. Date: Jul. 11, 2024

(54) NOVEL COMPOUNDS WITH DUAL INHIBITION ACTIVITY AGAINST SARS-COV-2 CRITICAL ENZYMES RDRP, MPRO AND HUMAN TMPRSS2

(71) Applicant: University of Sharjah, Sharjah (AE)

(72) Inventors: Sameh SOLIMAN, Sharjah (AE); Rania HAMDY, Sharjah (AE)

(21) Appl. No.: 18/088,285

(22) Filed: Dec. 23, 2022

(51) Int. Cl. A61K 31/706; A61K 31/352; A61P 31/14

(52) U.S. Cl. CPC A61K 31/706; A61K 31/352; A61P 31/14

(57) ABSTRACT

Novel SARS-CoV-2 inhibitors with dual targeting activity against both the viral RdRp and M^{pro} and host TMPRSS2 protease for therapeutic formulations and methods for treating SARS-CoV-2 infections. The present disclosure, in some embodiments, provides novel compounds with dual targeting activity against SARS-CoV-2. The compounds selectively target the viral RdRp and M^{pro} enzymes and host TMPRSS2 protease.

### TECHNICAL FIELD

The present invention relates to novel compounds with dual targeting activity against SARS-CoV-2. The compounds target both the viral RdRp and M^{pro} and host TMPRSS2 protease.

### BACKGROUND OF THE INVENTION

The outbreak due to COVID-19 caused by the spread of the novel coronavirus, severe acute respiratory syndrome coronavirus 2 (SARS-CoV-2), has posed an unprecedented threat to the global health and economy worldwide [1, 2]. More than 647 million confirmed cases have been diagnosed worldwide, causing over 6.65 million deaths [3]. The major concern associated with the virulence of SARS-CoV-2 is the improved immunity recognition escape mechanism. It evades the innate immune response mainly by the capping of spike protein with nsp16 [4] and the role of nsp1 [5] that suppresses the innate human response that both eventually leads to cytokine storm.

Although several vaccines were developed, the emerged mutant strains with higher transmissible rate may limit their efficacy and may endanger their future use [6]. Delta strain showed nine mutations within the targeted spike protein in many available vaccines that render less sensitivity to the neutralizing antibodies [7]. Omicron variant showed 30 mutations harbored within the spike protein [8]. The combinations of the FDA-approved monoclonal antibodies, such as tixagevimab plus cilgavimab that were effective in the neutralization of the early strains failed in vitro to neutralize the newly emerged strains [9].

Up to date, there is no available treatment, although several antivirals have been suggested. Hydroxychloroquine was the first repurposed FDA drug, which is stopped later by WHO [10]. Remdesivir has got emergency use authorization (EUA) as adaptive protocol for hospitalized COVID-19 patients [11]. Several antiviral drugs are currently in clinical trials such as favipiravir [12], AT-527 [13], niclosamide [14], and ritonavir/lopinavir [15]. Recently Pfizer commenced the clinical trial of PF-07321332 (nirmatrelvir) as a novel protease inhibitor. On October 2021, clinical trials conducted by Merk (NCT04575597) on Molnupiravir (development code EIDD-2801) [16] showed 50% reduced risk of COVID-19 death of high-risk patient including different variants [16, 17]. Both remdesivir and molnupiravir are RdRp inhibitors [18]. Furthermore, molnupiravir and paxlovid were granted the emergency use authorization EUA by FDA to manage the COVID-19 hospitalized patients [16, 17, 19]. Paxlovid is a combination therapy of nirmatrelvir and ritonavir that gain a EUA as COVID-19 medication [19].

Most of the aforementioned strategies are limited by their single targeting, particularly with the high probability of resistance due to evolved viral mutations. Therefore, there is an urgent need for the design of new inhibitors with multiple targeting as a therapeutic strategy to combat SARS-CoV-2 and possibly other emergent future viruses.

We rationalized the design of inhibitors with a dual activity that target both the SARS-CoV-2 RNA-dependent RNA polymerase (RdRp), required for viral replication [20] and human TMPRSS2, required for S protein activation and viral entry [21]. Dual targeting is a promising therapeutic approach compared to combination therapy as it reduces the toxicity and drug-drug interaction [22, 23], provides single-dose administration that is increasing the patient compliance, enhances the drug release, and provides an easy platform for future modifications to combat possible future outbreaks [24]. Several dual inhibitors drugs were used such as rivastigmine, asenapine, ladostigil, phenserine, clomipramine, amitriptyline, doxepin and desipramine [25]. A dual inhibitor approach has been developed against HIV [26] and Influenza virus [27] and showed great activity when compared to a single therapy [1]. Furthermore, the designed compounds possess basic moiety that initiates an alkaline pH, which disfavors the existence of SARS-CoV-2 in the human body and inactivates the protease enzyme [28].

### SUMMARY

The present disclosure, in some embodiments, provides novel compounds with dual targeting activity against SARS-CoV-2. The compounds selectively target the viral RdRp and M^{pro} enzymes and host TMPRSS2 protease.

In a first embodiment of the present disclosure, there is provided a compound with dual inhibition activity against SARS-CoV-2 including a coumarin or nucleoside analogue moiety including: (A) a 2H-chromen-6-yl, adenosine, cytosine, pyrimidine, isocytosine, azacytosine, triazine, or (2R,3R,4S,5R)-2-(4-Amino-pyrrolo[2,1-f]-[1,2,4]-triazin-7-yl)-3,4-dihydroxy-5-hydroxymethyl-tetrahydro-furan-2-carbonitrile; (B) a amino pyrimidine or guanidine analogue moiety including a 4-guanidino-benzamide, 4-guanidino-benzoic acid, or [1,2,4]-triazin-4-yl; and (C) a linker including an amide or an ester.

In a preferred embodiment of the present disclosure, there is provided a compound including (A) a nucleoside analogue moiety; (B) a guanidine analogue moiety; and (C) an amide linker according to formula RH11 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide) having the structure:

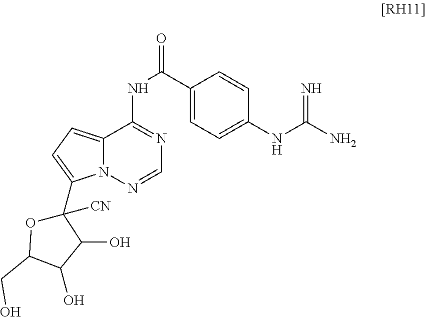 [SMILES: N#CC1(c2ccc3c(NC(=O)c4ccc(NC(=N)N)cc4)ncnn23)OC(CO)C(O)C1O]

In a preferred embodiment of the present disclosure, there is provided a compound including (A) a coumarin analogue; (B) a guanidine analogue moiety; and (C) an ester linker according to formula RH12 (4-guanidino-benzoic acid 4-isopropyl-2-oxo-2H-chromen-6-yl ester) having the structure:

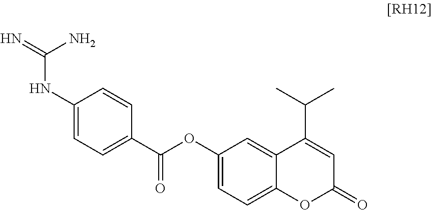 [SMILES: CC(C)c1cc(=O)oc2ccc(OC(=O)c3ccc(NC(=N)N)cc3)cc12]

In a preferred embodiment of the present disclosure, there is provided a compound including (A) a nucleoside analogue moiety; (B) a2-amino pyrimidine analogue; and (C) an amide linker according to formula RH13 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide) according to formula RH13 having the structure:

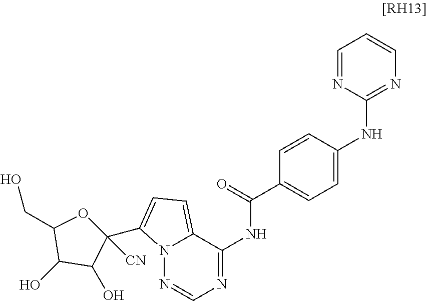 [SMILES: N#CC1(c2ccc3c(NC(=O)c4ccc(Nc5ncccn5)cc4)ncnn23)OC(CO)C(O)C1O]

In one aspect embodiment of the present disclosure, the compounds of the present disclosure exhibit dual targeting activity against SARS-CoV-2.

In one aspect of the present disclosure, the compounds of the present disclosure exhibit dual targeting activity against the human TMPRSS2 enzyme and the SARS-CoV-2 RNA-dependent RNA polymerase enzyme (RdRp).

In another aspect of the present disclosure, the compounds of the present disclosure exhibit dual targeting activity against the human TMPRSS2 enzyme and the SARS-CoV-2 RNA-dependent RNA polymerase (RdRp) enzyme and the M^{pro} enzyme.

In a preferred embodiment of the present disclosure, there is provided a pharmaceutical composition, comprising a therapeutically effective amount of any one of compounds RH11, RH12, or RH13, or pharmaceutically acceptable salts thereof, and one or more pharmaceutical excipients.

In a most preferred embodiment of the present disclosure, there is provided a method of treating a subject with SARS-CoV-2 infection, comprising administering to the subject in need thereof a therapeutically effective amount of any one of compounds RH11, RH12, or RH13 or pharmaceutically acceptable salt thereof, and one or more pharmaceutical excipients.

In one aspect of the present disclosure, there is provided a method of treating a subject with SARS-CoV-2 infection by administering to the subject a therapeutically effective amount of any one of compounds RH11, RH12, or RH13, where the subject is a mammal.

In one aspect of the present disclosure, there is provided a method of treating a subject with SARS-CoV-2 infection by administering to the subject a therapeutically effective amount of any one of compounds RH11, RH12, or RH13, where the mammal is a human.

In one aspect of the present disclosure, there is provided a method of treating a subject with SARS-CoV-2 infection by administering to the subject a therapeutically effective amount of any one of the compounds of the present invention, where the mammal is a human.

In a further aspect of the present disclosure, there is provided a kit for treating a subject with a SARS-CoV-2 infection, comprising any one of compounds RH11, RH12, or RH13, or a pharmaceutically acceptable salt thereof, and one or more pharmaceutical excipients.

While the disclosure is susceptible to various modifications and alternative forms, specific aspects thereof are shown byway of example in the drawing and will herein be described in detail. It should be understood, however, that the drawings and detailed description presented herein are not intended to limit the disclosure to the particular aspects disclosed, but on the contrary, the intention is to cover all modifications, equivalents, and alternatives falling within the spirit and scope of the present disclosure as defined by the appended claims.

Other features and advantages of this invention will become apparent in the following detailed description of preferred aspects of this invention, taken with reference to the accompanying drawings.

### BRIEF DESCRIPTION OF THE FIGURES

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows TMPRSS2 inhibitors mimicking the guanido group of the natural substrate of the cleavage site.

FIG. 2 provides RdRp inhibitors with nucleoside base analogues.

FIG. 3 provides a schematic for the rational design of novel dual inhibitors.

FIG. 4 provides (A) Synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide (RH11). (B) Synthesis of 4-guanidino-benzoic acid 4-isopropyl-2-oxo-2H-chromen-6-yl ester (RH12). Reagent and condition: (i) EDCI, pyridine, 0° C., overnight stirring, under N2.

FIG. 5 provides the synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide (RH13). Reagent and condition: (i) TBDMSCI, DMF, imidazole, DMAP. (ii) TBTU, DMF, DIPEA. (iii) TBAF, THF, room temperature, 4 h.

FIG. 6 provides the in vitro antiviral activity of compounds RH1-RH23.

FIG. 7 provides the selectivity indexes (CC50/IC50) of RH11-14, RH19 and RH20 compared to remdesivir.

FIG. 8 provides TMPRSS2 enzyme inhibitory activity. (A) Inhibition with RH11-14. (B) IC50 calculation of RH11 against TMPRSS2 enzyme. (C) IC50 calculation of RH12 against TMPRSS2 enzyme.

FIG. 9 provides SARS-CoV-2 RdRp enzyme inhibitory activity. (A) Inhibition with RH11-13 compared to remdesivir. (B) IC50 calculation of RH11 against SARS-CoV-2 RdRp enzyme. (C) IC50 calculation of RH12 against SARS-CoV-2 RdRp enzyme. (D) IC50 calculation of RH13 against SARS-CoV-2 RdRp enzyme.

FIG. 10 provides SARS-CoV-2 M^{pro} enzyme inhibitory activity. (A) Inhibition with RH11-14. (B) IC50 calculation of RH11 against M^{pro} enzyme. (C) IC50 calculation of RH12 against M^{pro} enzyme.

FIG. 11 provides the antiviral activity of RH11-RH13 compounds using a plaque reduction assay. The viral inhibition % for each mechanism was calculated based on the percentage of virus reduction.

FIG. 12 provides the molecular docking of compounds RH11 and RH12 with TMPRSS2 (PDB:20Q5) protein. (A) The interaction of RH11 within the active site of TMPRSS2. (B) The interaction of RH12 within the active site of TMPRSS2.

FIG. 13 provides the molecular docking of compounds RH11 and RH12 with RdRp (PDB:7BV2) protein. (A) The interaction of RH11 within the active site of RdRp. (B) The interaction of RH12 within the active site of RdRp.

FIG. 14 provides the molecular docking of compounds RH11 and RH12 with M^{pro} (PDB:6LU7) protein. (A) The interaction of RH11 within the active site of M^{pro}. (B) The interaction of RH12 within the active site of M^{pro}.

FIG. 15 provides the bioavailability radar compounds. The pink area indicated the preferred properties range of compounds (A) RH11, (B) RH12, (C) RH13, and (D) Remdesivir.

FIG. 16 provides the ¹H NMR of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide (RH11).

FIG. 17 provides the ¹³C NMR of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide (RH11).

FIG. 18 provides a conversion table where human equivalent dose (HED) dosage factors based on body surface area of other species are reported.

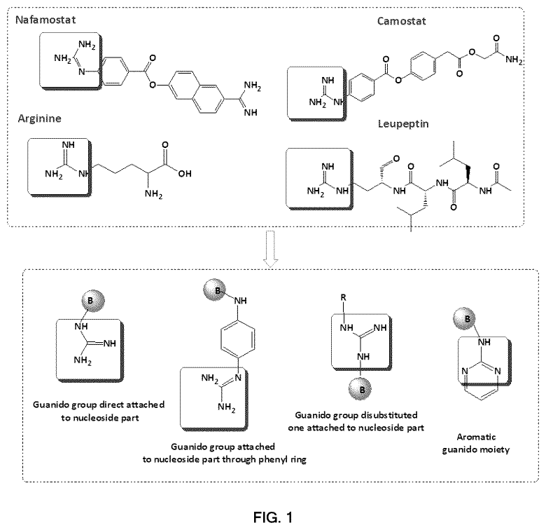

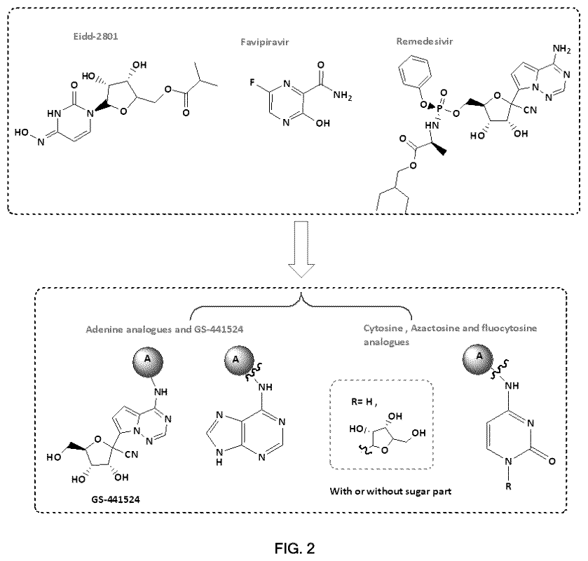

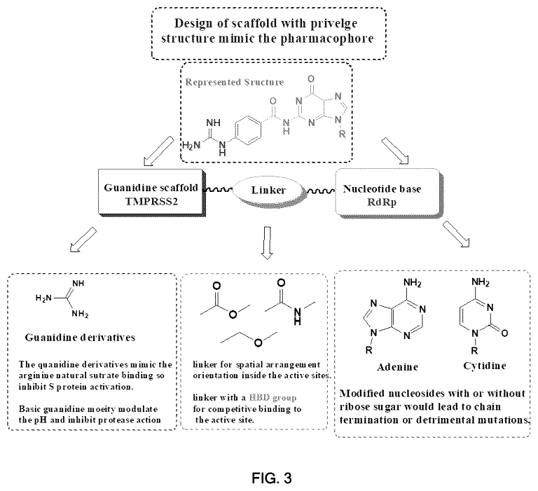

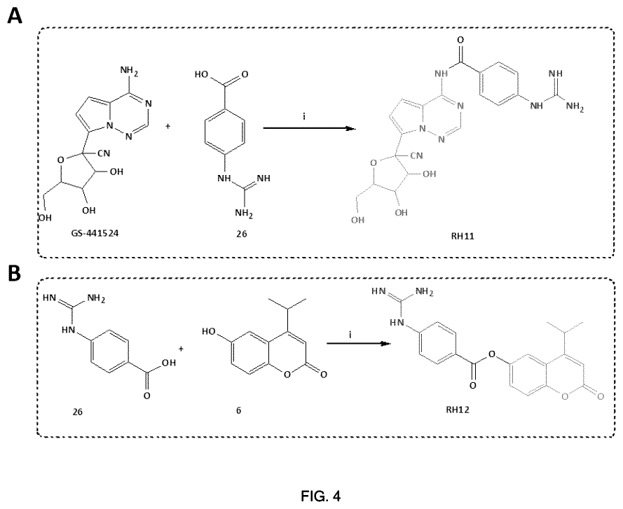

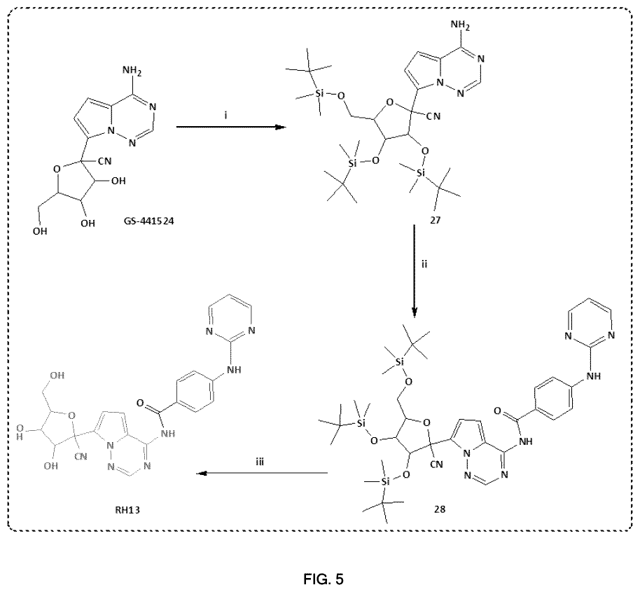

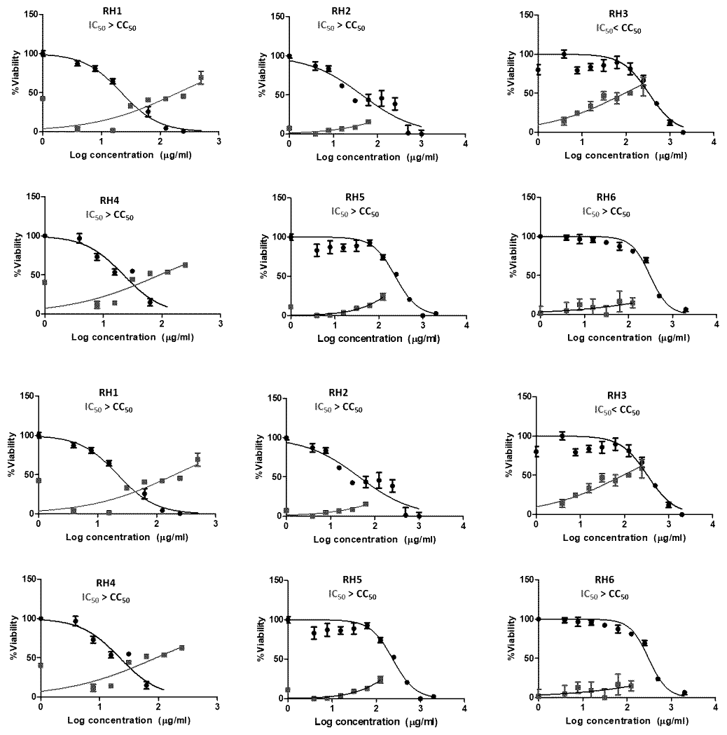

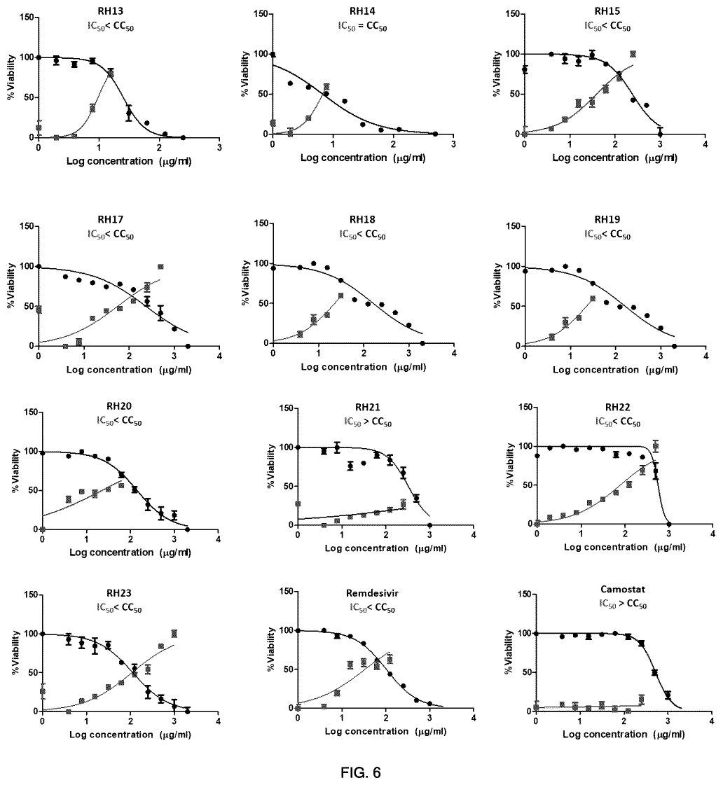

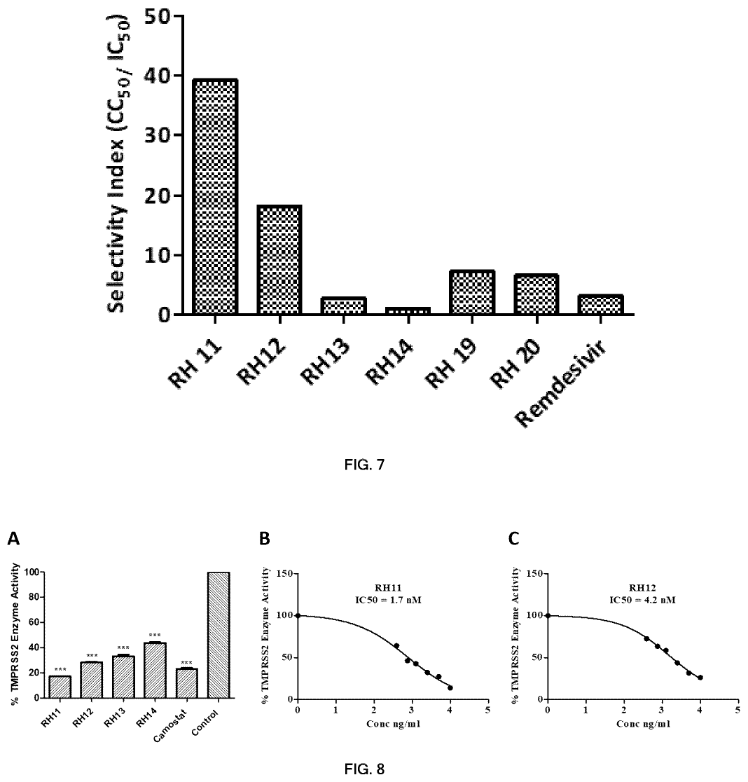

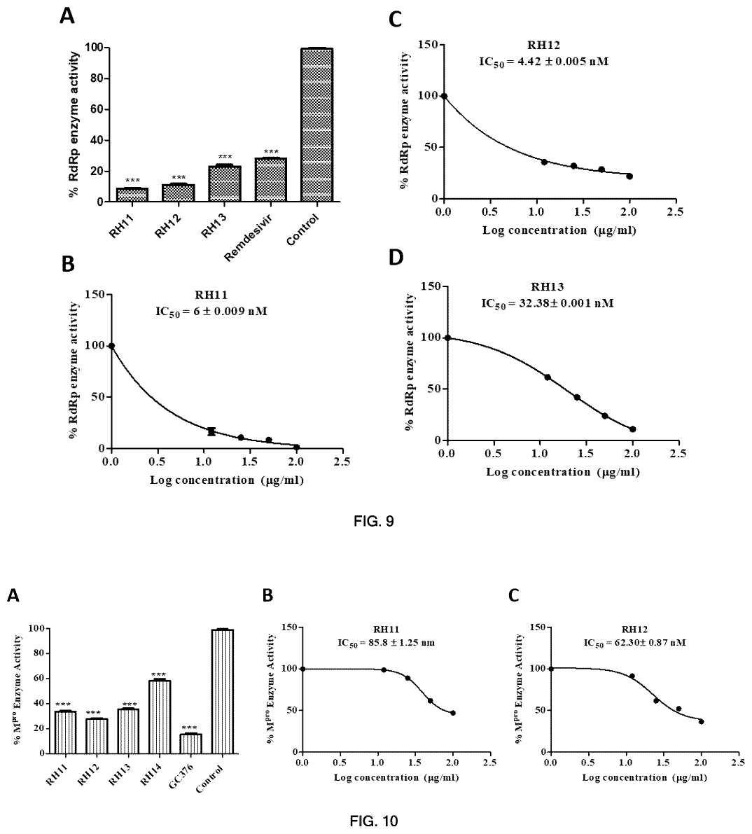

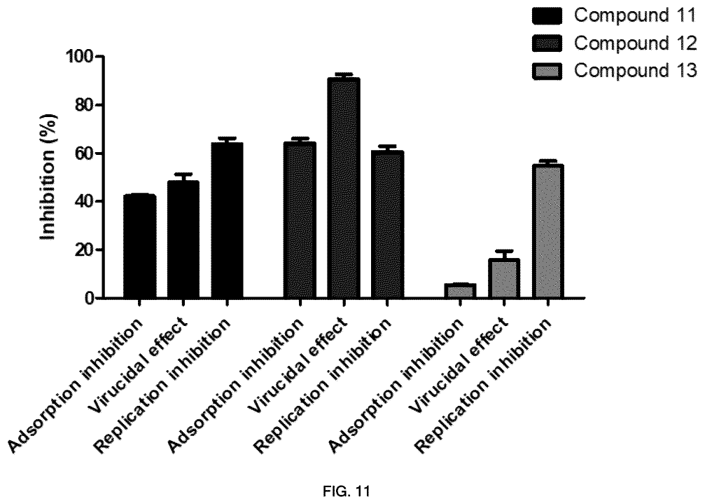

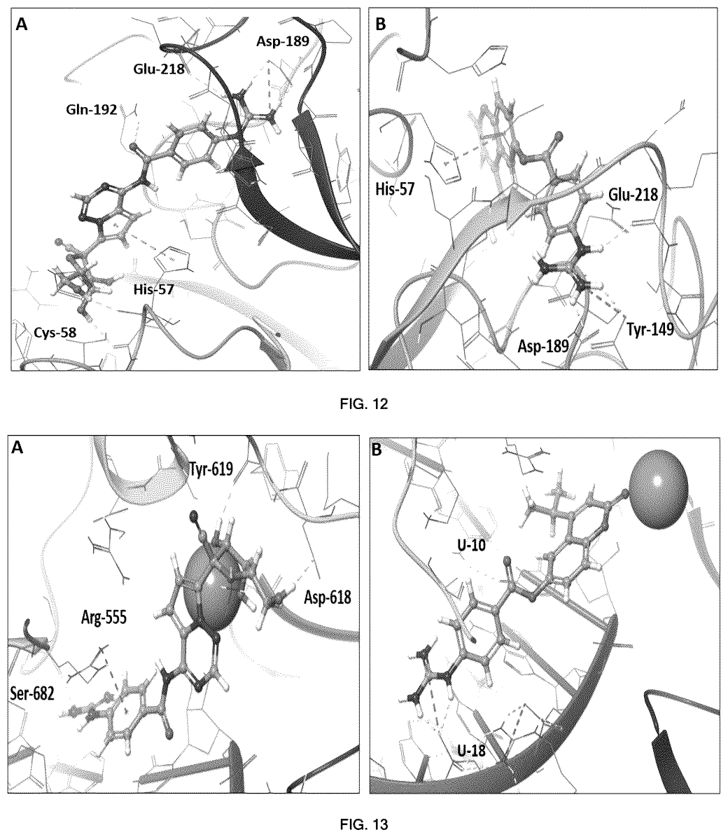

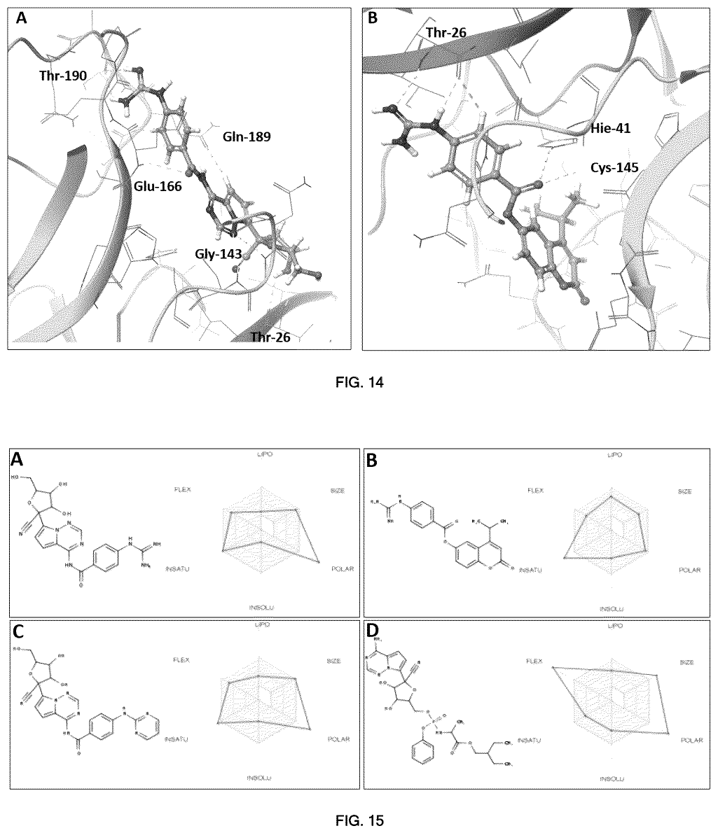

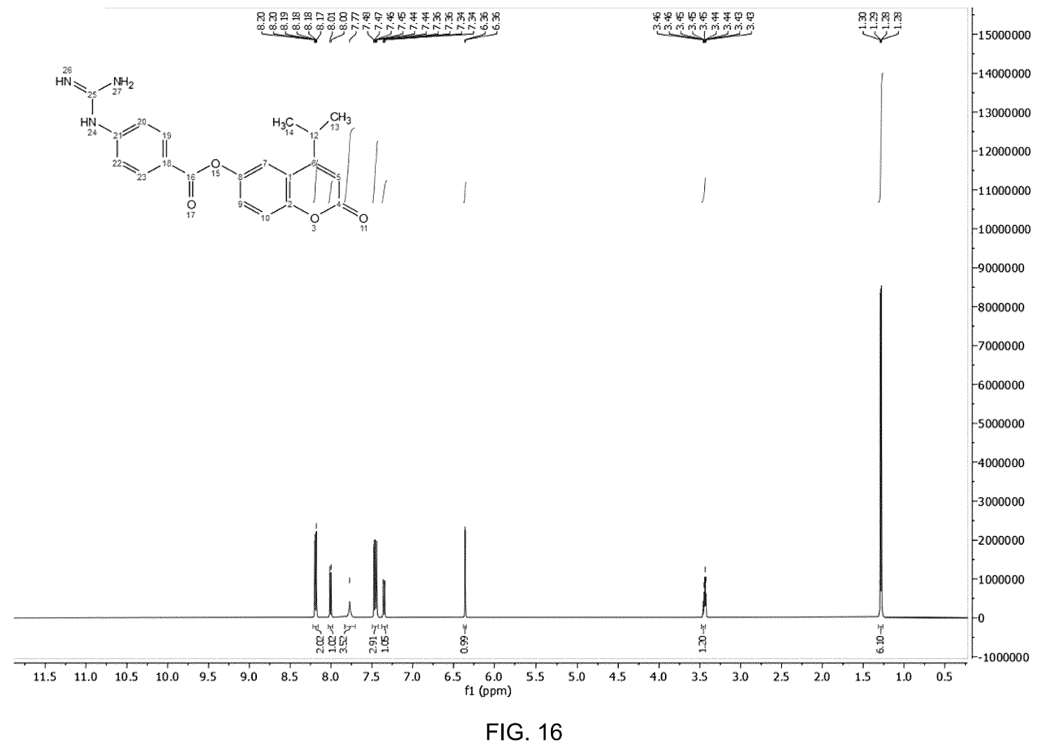

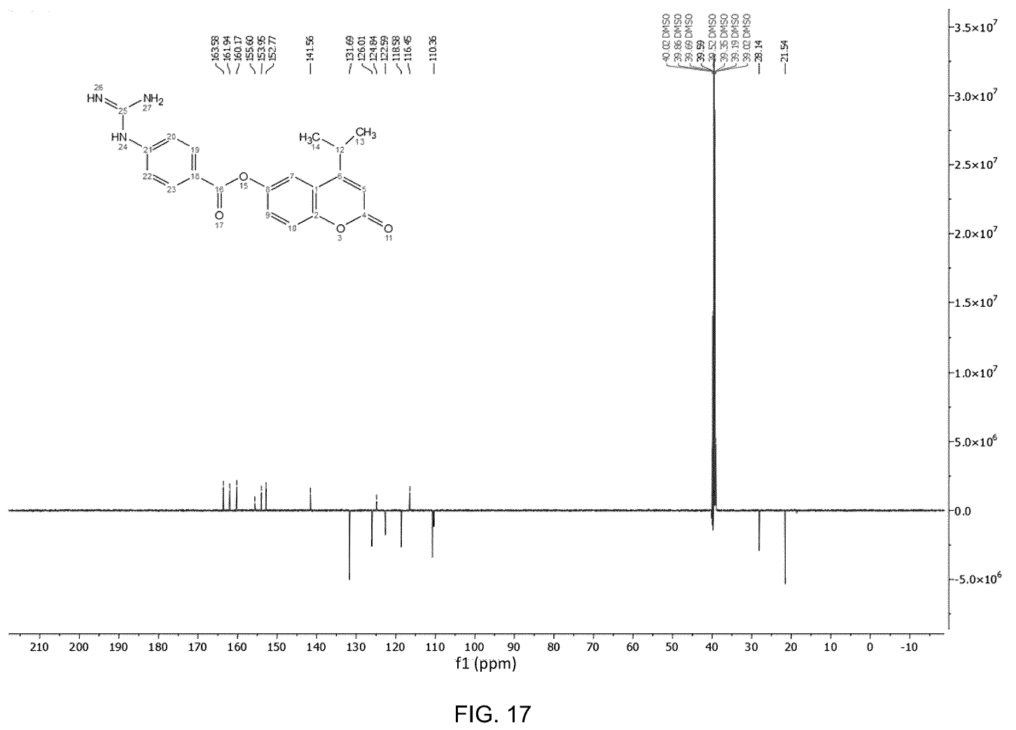

### DEFINITIONS

As used in this description and the accompanying claims, the following terms shall have the meanings indicated, unless the context otherwise requires:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “treatment” is used conventionally, e.g., the management or care of a subject for the purpose of combating, alleviating, reducing, relieving, improving, etc., one or more of the symptoms associated with SARS-CoV-2 infection. Administering effective amounts of the compound can treat one or more aspects of SARS-CoV-2 infection, including, but not limited to, inhibiting viral replication; reducing disease progression; stabilizing the disease; prolonging patient survival; enhancing patient's quality of life; reducing adverse symptoms associated with SARS-CoV-2 infection; and reducing the frequency, severity, intensity, and/or duration of any of the aforementioned aspects.

The term “subject” in accordance with the present invention, includes, e.g., mammals, such as dogs, cats, horses, rats, mice, monkeys, and humans.

As used herein, the term “therapeutically effective amount” means that amount of a drug or pharmaceutical agent that will elicit the biological or medical response of a tissue, system, animal or human that is being sought, for instance, by a researcher or clinician, and specifically indicates the amount of the compound which is effective to treat any symptom or aspect of SARS-CoV-2 infection. Effective amounts can be determined routinely. Further guidance on dosages and administration regimens is provided below. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Effective amounts can be determined routinely. Further guidance on dosages and administration regimens is provided below.

### DETAILED DESCRIPTION OF SPECIFIC EMBODIMENTS

A study was conducted to design inhibitors with a dual activity that target both the SARS-CoV-2 RNA-dependent RNA polymerase (RdRp), required for viral replication [20] and human TMPRSS2, required for S protein activation and viral entry [21]. Dual targeting is a promising therapeutic approach compared to combination therapy as it reduces the toxicity and drug-drug interaction [22, 23], provides single-dose administration that is increasing the patient compliance, enhances the drug release, and provides an easy platform for future modifications to combat possible future outbreaks [24]. Several dual inhibitors drugs were used such as rivastigmine, asenapine, ladostigil, phenserine, clomipramine, amitriptyline, doxepin and desipramine [25]. A dual inhibitor approach has been developed against HIV [26] and Influenza virus [27] and showed great activity when compared to a single therapy [1]. Furthermore, the designed compounds possess basic moiety that initiates an alkaline pH, which disfavors the existence of SARS-CoV2 in the human body and inactivates the protease enzyme [28].

We initiated a process starting by the identification of a potential therapeutic target for COVID-19 pandemic with high conservation regardless the emergence of variable variants. Early evidence emphasis that RdRp and M^{pro} were essential SARS-CoV-2 targets with high conservation rate [19]. Targeting these two enzymes is considered as a promising therapy against the current and any expected outbreaks [19].

The present invention discloses a series of dual inhibitor compounds with two scaffolds, a nucleoside analogue to target the RdRp [47], and a guanidine scaffold that mimic the TMPRSS2 substrate and inhibit its action [50]. Additionally, the basic feature of the guanidine scaffold disfavors the SARS-CoV-2 protease enzyme and enhance the antiviral activity [28]. The new series can provide an excellent platform for the drug design and development since it targets two major enzymes, RdRp and TMPRSS2, critical in SARS-CoV-2 infection. The dual inhibitors designed in this study incorporate adenosine, cytosine, isocytosine, azacytosine, triazine, and GS-441524, as nucleoside analogue, and coumarin scaffold, that is known to target SARS-CoV-2 RdRp enzyme [50]. The compounds of the present disclosure also incorporate free guanidine, free phenyl guanidine or substituted guanidine to target TMPRSS2.

RH11 is a modified structure of remdesivir incorporating a free phenyl guanidine moiety via amide linker that potentially improved the potency and the pharmacokinetics properties. RH12 is a coumarin-like structure that contains a free phenyl guanidine moiety via ester linker that showed a potent antiviral activity. Coumarins are known to exhibit dual antiviral activity against SARS-CoV-2 [49]. Furthermore, RH11 and RH12 compounds exhibited superior in vitro antiviral activity against SARS-CoV-2 at nM range compared to μM range of molnupiravir, an FDA approved drug. Molecular docking studies recognize the engagement of RH11 and RH12 in multiple interactions with the target proteins including RdRp, M^{pro} and TMPRSS2. Without being bound to any particular theory, the present invention proposes that MOA study revealed their potential activity by targeting the viral maturation cycle and its early invasion stage.

The designed compounds exhibited superior anti-SARS-CoV-2 activity with good physiochemical properties, bioavailability, and safety.

In a first embodiment of the present disclosure, there is provided a compound with dual inhibition activity against SARS-CoV-2 including a coumarin or nucleoside analogue moiety including: (A) a 2H-chromen-6-yl, adenosine, cytosine, pyrimidin, isocytosine, azacytosine, triazine, or (2R,3R,4S,5R)-2-(4-Amino-pyrrolo[2,1-f]-[1,2,4]-triazin-7-yl)-3,4-dihydroxy-5-hydroxymethyl-tetrahydro-furan-2-carbonitrile; (B) a 2-amino pyrimidine or guanidine analogue moiety including a 4-guanidino-benzamide, 4-guanidino-benzoic acid, or [1,2,4]-triazin-4-yl; and (C) a linker including an amide or an ester.

In a preferred embodiment of the present disclosure, there is provided a compound including (A) a nucleoside analogue moiety; (B) a guanidine analogue moiety; and (C) an amide linker according to formula RH11 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide) having the structure:

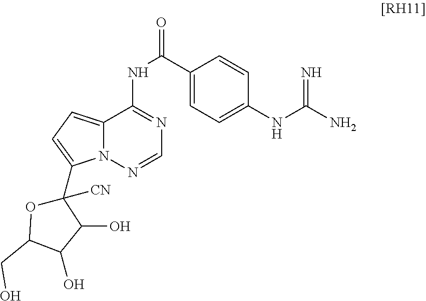 [SMILES: N#CC1(c2ccc3c(NC(=O)c4ccc(NC(=N)N)cc4)ncnn23)OC(CO)C(O)C1O]

In a preferred embodiment of the present disclosure, there is provided a compound including (A) a coumarin; (B) a guanidine analogue moiety; and (C) an ester linker according to formula RH12 (4-guanidino-benzoic acid 4-isopropyl-2-oxo-2H-chromen-6-yl ester) having the structure:

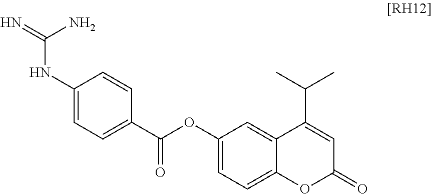 [SMILES: CC(C)c1cc(=O)oc2ccc(OC(=O)c3ccc(NC(=N)N)cc3)cc12]

In a preferred embodiment of the present disclosure, there is provided a compound including (A) a nucleoside analogue moiety; (B) a 2-amino pyrimidine analogue; and (C) an amide linker according to formula RH13 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide) according to formula RH13 having the structure:

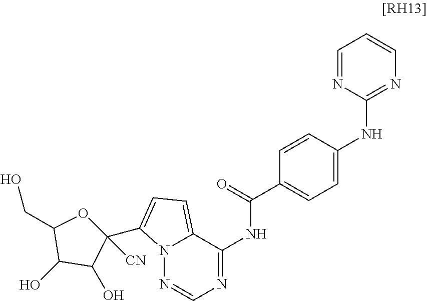 [SMILES: N#CC1(c2ccc3c(NC(=O)c4ccc(Nc5ncccn5)cc4)ncnn23)OC(CO)C(O)C1O]

In one aspect embodiment of the present disclosure, the compounds of the present disclosure exhibit dual targeting activity against SARS-CoV-2.

In one aspect of the present disclosure, the compounds of the present disclosure exhibit dual targeting activity against the human TMPRSS2 enzyme and the SARS-CoV-2 RNA-dependent RNA polymerase enzyme (RdRp).

In another aspect of the present disclosure, the compounds of the present disclosure exhibit dual targeting activity against the human TMPRSS2 enzyme and the SARS-CoV-2 RNA-dependent RNA polymerase (RdRp) enzyme and the M^{pro} enzyme.

In a preferred embodiment of the present disclosure, there is provided a pharmaceutical composition, comprising a therapeutically effective amount of any one of compounds RH11, RH12, or RH13, or pharmaceutically acceptable salts thereof, and one or more pharmaceutical excipients.

In a most preferred embodiment of the present disclosure, there is provided a method of treating a subject with SARS-CoV-2 infection, comprising administering to the subject in need thereof a therapeutically effective amount of any one of compounds RH11, RH12, or RH13 or pharmaceutically acceptable salt thereof, and one or more pharmaceutical excipients.

In one aspect of the present disclosure, there is a provided method of treating a subject with SARS-CoV-2 infection by administering to the subject a therapeutically effective amount of any one of the compounds RH11, RH12, or RH13, where the subject is a mammal.

In one aspect of the present disclosure, there is provided a method of treating a subject with SARS-CoV-2 infection by administering to the subject a therapeutically effective amount of any one of compounds RH11, RH12, or RH13, where the mammal is a human.

In one aspect of the present disclosure, there is provided a method of treating a subject with SARS-CoV-2 infection by administering to the subject a therapeutically effective amount of any one of the compounds of the present invention, where the mammal is a human.

The compounds described in the present disclosure show dual enzyme inhibition activity and anti-viral activity against SARS-CoV-2. The results reported herein show these compounds can be useful in the treatment of SARS-CoV-2. Consequently, successful protocols can be translated for therapy of these patients.

Compositions featuring the aforementioned compounds may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin. propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations may conveniently be presented in unit dosage form and may be prepared by any methods well known in the art of pharmacy. The amount of compound which can be combined with a carrier material to produce a single dosage form will vary depending upon the subject being treated, the particular mode of administration. The amount of an active ingredient which can be combined with a carrier material to produce a single dosage form will usually be that amount of the compound which produces a therapeutic effect. Usually, out of one hundred percent, this amount will range from about 1 wt % to about 99 wt % of active ingredient, preferably from about 5 wt % to about 70 wt %, most preferably from about 10 wt % to about 30 wt %.

In certain embodiments, a formulation of the compound includes an excipient selected from the group consisting of cyclodextrins, liposomes, micelle forming agents, e.g., bile acids, and polymeric carriers, e.g., polyesters and polyanhydrides; and an active ingredient that may be the compound and/or one of its pharmaceutically acceptable derivatives. In certain embodiments, an aforementioned formulation renders orally bioavailable a compound or its derivative.

Methods of preparing these formulations or compositions include the step of bringing into association the compound with the carrier and, optionally, one or more accessory ingredients. Usually, the formulations are prepared by uniformly and intimately bringing into association a compound of the present invention with liquid carriers, or finely divided solid carriers, or both, and then, if necessary, shaping the product.

Liquid dosage forms for oral administration of the compound include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A formulation of the compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also include buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

The tablets, and other solid dosage forms of the formulation of the compound, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations of the pharmaceutical compositions of the compound for rectal or vaginal administration may be presented as a suppository, which may be prepared by the compound with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of the compound include powders, sprays, ointments, pastes, creams, lotions, gels, solutions, patches and inhalants. The extract may be mixed under sterile conditions with a pharmaceutically-acceptable carrier, and with any preservatives, buffers, or propellants which may be required. The ointments, pastes, creams and gels may contain, in addition to an extract, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an extract, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Powders and sprays can contain, in addition to an extract, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compound to the body. Such dosage forms can be made by dissolving or dispersing an extract in the proper medium. Absorption enhancers can also be used to increase the flux of the extract or dispersing the extract in a polymer matrix or gel.

Pharmaceutical compositions suitable for parenteral administration include one or more components of the compound in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compound may be formulated into pharmaceutically-acceptable dosage forms by conventional methods known to those of skill in the art. The compound may be formulated for administration in any convenient way for use in human or veterinary medicine, by analogy with other pharmaceuticals.

### Methods of Treatment of SARS-CoV-2 Infection

The above compound compositions may be used in novel therapeutic methods of treatment in patients afflicted by SARS-CoV-2 infection. The methods include administering to a subject an effective amount of a pharmaceutical compound composition. In representative embodiments, the subject suffers from SARS-CoV-2 infection. In specific embodiments, the SARS-CoV-2 infection is asymptomatic or symptomatic with different degrees of severity.

The above invention can be used to treat SARS-CoV-2 irrespective of the type of strain, and irrespective of the severity associated with the infection, including, but not limited to moderately symptomatic to severely symptomatic can also be treated.

As anticipated above, the compound may be administered by any appropriate route, for example orally, parenterally, topically, or rectally. It will be appreciated that the preferred route may vary with, for example, the condition of the recipient of the compound and the cancer to be treated. In certain embodiments, the extract may be especially suitable for the preparation of pharmaceuticals for intravenous administration, such as intravenous injection or infusion, provided that it does not contain components with serum-precipitating and/or hemagglutinating properties which disturb such an application. The extract may therefore be provided in the form of ampoule preparations which are directed to intravenous administration. In still other embodiments, the method comprises systemic administration of a subject composition to a subject.

Exemplary doses of the compound in the range from about 0.001, 0.01, 0.1, 0.5, 1, 10, 15, 20, 25, 50, 100, 200, 300, 400, 500, 600, or 750 to about 1000 mg/day per kg body weight of the subject. In certain embodiments, the dose of the compound will typically be in the range of about 100 mg/day to about 1000 mg/day per kg body weight of the subject, specifically in the range of about 200 mg/day to about 750 mg/day per kg, and more specifically in the range of about 250 mg/day to about 500 mg/day per kg. In an embodiment, the dose is in the range of about 50 mg/day to about 250 mg/day per kg. In a further embodiment, the dose in the range of about 100 mg/day to about 200 mg/day per kg. In an embodiment, the dose is in the range of about 15 mg/day to 60 mg/day per kg. In a further embodiment, the dose is in the range of about 20 mg/day to 50 mg/day per kg. In an additional embodiment, the dose is in the range of about 25 mg/day to 45 mg/day per kg.

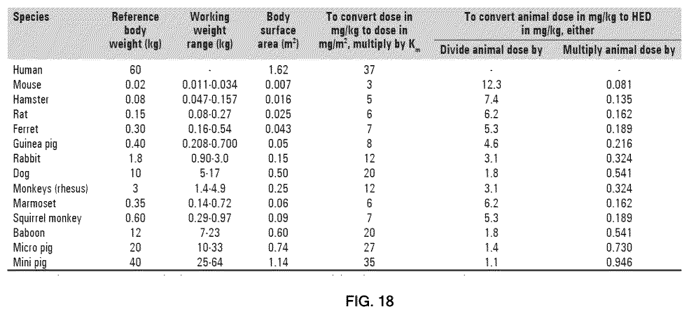

The data obtained from cell culture assays and animal studies may be used in formulating a range of dosage for use in humans. For example, effective dosages achieved in one animal species may be extrapolated for use in another animal, including humans, as illustrated in the conversion table of FIG. 18 where human equivalent dose (HED) dosage factors based on body surface area of other species are reported [51]. The dosage of any supplement, or alternatively of any components therein, lies preferably within a range of circulating concentrations that include the ED₅₀ with little or no toxicity. The dosage may vary within this range depending upon the dosage form employed and the route of administration utilized. For the compound or combinations of the compound and other chemotherapeutic agents, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In a further aspect of the present disclosure, there is provided a kit for treating a subject afflicted by SARS-CoV-2, the kit comprising any one of compounds RH11, RH12, or RH13, a pharmaceutically acceptable salt thereof, and one or more pharmaceutical excipients.

The present invention provides kits for novel therapeutic methods in COVID-19 patients. For example, a kit may include one or more pharmaceutical compositions of the compound as described above. The compositions may be pharmaceutical compositions comprising a pharmaceutically acceptable excipient. In other embodiments involving kits, this invention provides a kit including the compound, optionally a chemotherapeutic agent, and optionally instructions for their use in the treatment of SARS-CoV-2. In still other embodiments, the invention provides a kit comprising one more pharmaceutical composition and one or more devices for accomplishing administration of such compositions. For example, a subject kit may comprise a pharmaceutical composition and catheter for accomplishing direct intraarterial injection of the composition in a patient with SARS-CoV-2 infection. In an embodiment, the device is an intraarterial catheter. Such kits may have a variety of uses, including, for example, therapy, diagnosis, and other applications.

### Materials and Methods

### Chemistry

Reactions were monitored by thin-layer chromatography (TLC) using pre-coated silica gel plates (Kieselgel 60 F254, BDH, Taufkirchen, Germany), and spots were visualized with UV light at 254 nm. Melting points (mp) were determined using a Gallenkamp melting point apparatus (London, UK). 1HNMR spectra were recorded on a Bruker spectrometer at 500 MHz. Chemical shifts were expressed in parts per million (ppm) relative to TMS and coupling constant (J) values were represented in hertz (Hz). The signals were designated as follows: s, singlet; d, doublet; t, triplet; m, multiplet. Mass spectroscopic data were obtained through positive electrospray ionization (ESI) mass spectrum (Bruker Daltonics mass spectrometer, Bremen, Germany).

### General Procedure for Amide Coupling

Synthesis of intermediate amides was achieved by stirring 4-nitrobenzoyl chloride with different nucleoside analogues under nitrogen at 0° C. in pyridine and anhydrous chloroform followed by extraction with ethyl acetate:water (3:1) to afford the corresponding amide.

### Reduction of Nitro-Containing Compounds

Nitro compounds were reduced to the corresponding amine using Pd/C 10% in anhydrous THE and bubbled with the hydrogen gas for 3-16 h. The mixture was then filtered through celite and evaporated to dryness for further purification.

### Synthesis of 6-(4-amino-6-p-tolylamino-[1.3.5]-triazin-2-ylmethoxy)-4-isopropyl-chromen-2-one (RH1)

### Synthesis of 6-chloromethyl-N-o-tolyl-[1,3,5]-triazine-2,4-diamine (3)

First, o-phenylbiguanide (1, 10 mmol) was dissolved in methanol (10 mL), then ethyl bromoacetate (2, 10 mmol) was added dropwise. The reaction mixture was stirred at room temperature for overnight. Methanol was removed by evaporation under vacuum. The residue was purified by flash chromatography using ethyl acetate:hexane (3:2) to give the desired product in moderate yield (73%). ¹H-NMR (DMSO-d6): δ 2.19 (s, 3H, CH3), 4.28 (s, 2H, CH2), 7.03 (s, 2H, NH2), 7.07-7.10 (m, 1H, ArH), 7.14-7.17 (m, 1H, ArH), 7.21 (d, 1H, J=7.5, ArH), 7.31 (d, 1H, J=7.5, ArH). MS analysis for C₁₁H₁₂ClN₅: Calcd mass: 249.08, found (m/z, ES+): 250.

### Synthesis of 7-hydroxy-4-isopropylcoumarin (6)

To a mixture of resorcinol (5, 10 mmol) and ethyl isobutyryl acetate (4.15 mmol), excess H2SO4 (98%) was slowly added with stirring at 0° C. for 1 h. The mixture was then stirred at room temperature until the reaction (monitored by TLC) was completed. The mixture was poured into ice water and left overnight. The precipitated solid was filtered, washed, neutralized with water, and purified with crystallization from dioxane. Yield 74%. mp 173-174° C. ¹H-NMR (DMSO-d6): δ 1.22 (d, 6H, 2CH3 of isopropyl), 3.29 (m, 1H, CH of isopropyl), 6.07 (s, 1H, ArH), 6.71 (d, 1H, J=2.5, ArH), 6.80 (dd, 1H, J=9, ArH), 7.69 (d, 1H, J=8, ArH), 10.54 (s, 1H, OH). MS analysis for C₁₂H₁₂O₅: Calcd mass: 204.08, found (m/z, ES+): 205.09.

### Synthesis of 6-(4-amino-6-o-tolylamino-[1,3,5]-triazin-2-ylmethoxy)-4-isopropyl-chromen-2-one (RH1)

A mixture of 6-chloromethyl-N-o-tolyl-[1,3,5]-triazine-2,4-diamine (3, 10 mmol), 7-hydroxy-4-isopropylcoumarin (6, 10 mmol) and potassium carbonate (3 gm) in anhydrous dioxane (15 mL) was refluxed for 5 h. After cooling, the precipitate was collected and purified with crystallization from dioxane. Yield 62%. 1H-NMR (DMSO-d6): A mixture of 6-chloromethyl-N-o-tolyl-[1,3,5]-triazine-2,4-diamine (3, 10 mmol), 7-hydroxy-4-isopropylcoumarin (6, 10 mmol) and potassium carbonate (3 gm) in anhydrous dioxane (15 mL) was refluxed for 5 h. After cooling, the precipitate was collected and purified with crystallization from dioxane. Yield 62%. ¹H-NMR (DMSO-d6): δ 1.22 (d, 6H, 2 CH3 of isopropyl), 2.15 (s, 3H, CH3), 3.29 (m, 1H, CH of isopropyl), 4.92 (s, 2H, CH2), 6.93 (s, 2H, NH2), 6.94-6.97 (m, 3H, ArH), 7.05 (d, 2H, J=6.5, ArH), 7.17 (d, 1H, J=7.4, ArH), 7.25 (d, 1H, J=6.85, ArH), 7.79 (d, 1H, J=8.4, ArH), 8.87 (s, 1H, NH). ¹³C-NMR (DMSO-d6): δ 18.37, 21.91, 30.46, 65.11, 102.05, 106.68, 112.01, 112.59, 123.89, 125.87, 126.51, 130.60, 133.38, 136.83, 154.96, 160.75, 161.26, 162.57, 165.09, 165.99, 166.96, 172.70. MS analysis for C₂₃H₂₃N₅O₃: Calcd mass: 417.18, found (m/z, ES+): 418.26.

### Synthesis of N-(4-isopropyl-6-oxo-1,6-dihydro-pyrimidin-2-yl)-N′-o-tolyl-guanidine (RH2)

A solution of N-p-tolyl phenyl biguanide (1, 10 mmol) and ethyl isobutyrylacetate (4, 10 mmol) in ethanol (15 mL) was heated at 50° C. for 18 h. The formed precipitate was filtered and crystalized from dioxane. Yield 70%. ¹H-NMR (DMSO-d6): d 1.12 (d, 6H, 2 CH3 of isopropyl), 2.24 (s, 3H, CH3), 2.54 (m, 1H, CH of isopropyl), 5.53 (s, 1H, CH), 7.11 (t, 1H, J=8, ArH), 7.19 (t, 1H, J=7.5, ArH), 7.25 (d, 2H, J=8.5, ArH), 7.52 (s, 1H, NH), 9.34 (s, 1H, NH), 10.87 (s, 1H, NH). ¹³C-NMR (DMSO-d6): δ 17.87, 21.29, 36.24, 101.94, 124.53, 124.83, 126.69, 130.58, 132.33, 134.52, 157.50, 158.83, 164.39, 171.49. MS analysis for C₁₅H₁₉N₅O: Calcd mass: 285.16, found (m/z, ES+): 286.

### Synthesis of N-(2-oxo-1,2-dihydro-pyrimidin-4-yl)-4-(pyrimidin-2-ylamino)-benzamide (RH3)

### Synthesis of 4-(pyrimidin-2-ylamino)-benzoic acid (9)

A mixture of p-amino-benzoic acid (PABA, 7, 10 mmol) and 2-chloropyrimidine (8, 10 mmol) in 20 mL aqueous HCl was stirred at room temperature for 24 h. The formed precipitate was filtered, washed with NaHCO₃ and purified with column chromatography using petroleum ether:ethyl acetate (4:1). Yield (67%). ¹H-NMR (DMSO-d6): δ 6.93 (t, 1H, J=5.5, ArH), 7.85-7.90 (m, 4H, ArH), 8.55 (d, 2H, J=5.5, ArH), 10.4 (s, 1H, NH), 12.5 (s, 1H, COOH). MS analysis for C₁₁H₉N₃O₂: Calcd mass: 215.07, found (m/z, ES+): 216.09.

### Synthesis of N-(2-oxo-1,2-dihydro-pyrimidin-4-yl)-4-(pyrimidin-2-ylamino)-benzamide (RH3)

The 4-(pyrimidin-2-ylamino)-benzoic acid (9, 2 mmol) was dissolved in 10 mL anhydrous pyridine under inert condition. HOBT (6 mmol), DIPEA (4 mmol), EDCI (6 mmol) and cytosine (10, 3 mmol) were added sequentially and stirred at room temperature overnight and concentrated under vacuum for further purification with preparative TLC using methanol:chloroform (4:1). Yield (63%). ¹H-NMR (DMSO-d6): d 6.2 (s, 2H, NH), 6.88 (t, 1H, J=6.5, ArH), 7.28 (d, 2H, J=7.5, ArH), 7.81 (d, 2H, J=8, ArH), 7.88 (m, 2H, ArH), 8.49 (d, 2H, J=4.5, ArH), 9.84 (s, 1H, NH). ¹³C-NMR (DMSO-d6): δ 112.85, 116.38, 117.55, 117.98, 126.98, 129.77, 130.29, 132.08, 141.31, 158.84, 170.08, 171.78. MS analysis for C₁₅H₁₂N₆O₂: Calcd mass: 308.10, found (m/z, ES+): 309.30.

### Synthesis of 2-(4-amino-6-o-tolylamino-[1.3.5]-triazin-2-ylmethyl)-4-methyl-2H-phthalazin-1-one (RH4)

### Synthesis of 4-methyl-2H-phthalazin-1-one (12)

Hydrazine hydrate (10 mmol) was added to a solution of 2-acetylbenzoic acid (11, 10 mmol) in 15 mL ethanol. The reaction mixture was refluxed for 14 h. The precipitate was filtered and washed with ethanol. Yield (81%). ¹H-NMR (DMSO-d6): d 2.72 (s, 3H, CH3), 7.85-7.93 (m, 2H, ArH), 8.01-8.07 (m, 1H, ArH), 8.5-8.52 (m, 1H, ArH), 9.5 (s, 1H, NH). MS analysis for C₉H₈N₂O: Calcd mass: 160.07, found (m/z, ES+): 161.03.

### Synthesis of 2-(4-amino-6-o-tolylamino-[1,3,5]-triazin-2-ylmethyl)-4-methyl-2H-phthalazin-1-one (RH4)

A mixture of 4-methyl-2H-phthalazin-1-one (12, 5 mmol), 6-chloromethyl-N-o-tolyl-[1,3,5]-triazine-2,4-diamine (3, 6 mmol), potassium carbonate (15 mmol) and anhydrous dioxane (12 mL) was heated under reflux for 6 h. After cooling at room temperature, the precipitate was filtered and washed with anhydrous dioxane. The filtrate was evaporated under reduced pressure and the solid obtained was purified with flash chromatography using methanol:chloroform (1:9) as eluent. Yield 74%. ¹H-NMR (DMSO-d6): δ 2.28 (s, 3H, CH3), 2.52 (s, 3H, CH3), 5.06 (s, 2H, CH2), 6.82 (s, 2H, NH2), 7.20 (d, 2H, J=8, ArH), 7.31 (d, 2H, J=7.5, ArH), 7.87-7.89 (m, 1H, ArH), 7.97 (d, 2H, J=5, ArH), 8.26 (d, 1H, J=8, ArH), 8.63 (s, 1H, NH). ¹³C-NMR (DMSO-d6): δ 21.19, 25.19, 76.33, 115.36, 122.82, 124.31, 128.11, 129.61, 130.76, 136.42, 140.26, 147.05, 148.27, 149.31, 150.10, 157.34, 164.27, 165.86, 167.82. MS analysis for C₂₀H₁₉N₇O: Calcd mass: 373.17, found (m/z, ES+): 374.

### Synthesis of N-(9H-purin-6-yl)-4-(pyrimidin-2-ylamino)-benzamide (RH5)

### Synthesis of 4-nitro-N-(9H-purin-6-yl)-benzamide (15)

The 4-nitro benzoyl chloride (14, 10 mmol) in anhydrous chloroform was added dropwise to a cooled solution of adenine (13, 10 mmol) in 10 mL pyridine and heated at 60° C. overnight. The solvent was removed under vacuum and dissolved in methanol (20 mL). The pH was adjusted to 9 with 3M NaOH solution. The formed precipitate was filtered and recrystallized from ethanol. Yield (63%). ¹H-NMR (DMSO-d6): δ 8.32 (d, 2H, J=7.5, ArH), 840 (d, 2H, J=8, ArH), 8.54 (s, 1H, ArH), 8.75 (s, 1H, ArH), 11.93 (s, 1H, NH), 12.5 (s, 1H, NH). MS analysis for C₁₂H₈N₆O₃: Calcd mass: 284.07, found (m/z, ES+): 285.

### Synthesis of 4-amino-N-(9H-purin-6-yl)-benzamide (16)

The (4-nitro-phenyl)-9H-purin-6-yl-benzamide (15) was reduced to the corresponding amine using Pd/C 10% in anhydrous THE (10 mL) and bubbled with the hydrogen gas. The mixture was filtered through celite and evaporate to dryness for further purification. ¹H-NMR (DMSO-d6): δ 6.02 (s, 2H, NH2), 6.61 (d, 2H, J=8.5, ArH), 7.87 (d, 2H, J=8.5, ArH), 8.41 (s, 1H, ArH), 8.66 (s, 1H, ArH), 10.93 (s, 1H, NH), 12.22 (s, 1H, NH). MS analysis for C₁₂H₁₀N₆O: Calcd mass: 254.09, found (m/z, ES+): 255.11.

### Synthesis of N-(9H-Purin-6-yl)-4-(pyrimidin-2-ylamino)-benzamide (RH5)

A mixture of 4-amino-N-(9H-purin-6-yl)-benzamide (12, 10 mmol) and 2-chloropyrimidine (8, 10 mmol) in 20 mL aqueous HCl was heated at 60° C. for 24 h. The formed precipitate was filtered, washed with NaHCO₃ and recrystallized from ethanol. Yield (57%). ¹H-NMR (DMSO-d6): δ 5.93 (t, 1H, J=6.5, ArH), 7.34 (d, 2H, J=7.5, ArH), 7.82-7.93 (m, 4H, ArH), 8.55 (d, 2H, J=6.5 ArH), 10.93 (s, 2H, NH), 12.28 (s, 1H, NH). 13C-NMR (DMSO-d6): 113.38, 115.40, 117.55, 122.04, 129.27, 130.28, 144.76, 156.04, 157.01, 159.64, 161.26, 167.16. MS analysis for C₁₆H₁₂N₈O: Calcd mass: 332.11, found (m/z, ES+): 333.24.

### Synthesis of N-(4-Methyl-pyridin-2-yl)-4-(pyrimidin-2-ylamino)-benzamide (RH6)

### Synthesis of N-(4-methyl-pyridin-2-yl)-4-nitro-benzamide (18)

The 4-nitro benzoyl chloride (14, 10 mmol) in anhydrous chloroform was added dropwise to a cooled solution of 4-methyl-pyridin-2-ylamine (17, 10 mmol) in 10 mL pyridine and stirred at 0° C. for 3 h. The formed precipitate was filtered and recrystallized from dioxane. Yield (62%). 1H-NMR (DMSO-d6): δ 2.37 (s, 3H, CH3), 8.04 (d, 1H, J=5, ArH), 8.20 (d, 2H, J=7.5, ArH), 8.26-8.28 (m, 2H, ArH), 8.33 (d, 2H, J=7, ArH), 11.08 (s, 1H, NH). MS analysis for C13H11N3O3: Calcd mass: 257.08, found (m/z, ES+): 258.

### Synthesis of 4-amino-N-(4-methyl-pyridin-2-yl)-benzamide (19)

The N-(4-methyl-pyridin-2-yl)-4-nitro-benzamide (18, 10 mmol) was reduced to the corresponding amine using Pd/C 10% in anhydrous THE (10 mL) and bubbled with hydrogen gas. The mixture was filtered through celite and evaporated to dryness for further purification. Yield (72%). ¹H-NMR (DMSO-d6): δ 2.37 (s, 3H, CH3), 6.02 (s, 2H, NH2), 8.24 (d, 2H, J=7.5, ArH), 8.48 (d, 2H, J=7, ArH), 8.5-8.52 (m, 2H, ArH), 9.42 (d, 1H, J=2, ArH), 11.8 (s, 1H, NH). MS analysis for C₁₃H₁₁N₃O₃: Calcd mass: 227.11, found (m/z, ES+): 228.

### Synthesis of N-(4-methyl-pyridin-2-yl)-4-(pyrimidin-2-ylamino)-benzamide (RH6)

A mixture of 4-amino-N-(4-methyl-pyridin-2-yl)-benzamide (19, 10 mmol) and 2-chloropyrimidine (8, 10 mmol) in 20 mL aqueous HCl was stirred for 36 h. The formed precipitate was filtered, washed with NaHCO₃, and recrystallized from ethanol. Yield (57%). ¹H-NMR (DMSO-d6): δ 2.37 (s, 3H, CH3), 6.89 (s, 1H, NH), 7.03 (t, 1H, J=5.5, ArH), 7.87 (d, 1H, J=7, ArH), 8.11 (d, 2H, J=6.5, ArH), 8.17-8.22 (m, 4H, ArH), 8.62 (d, 2H, J=7.5, ArH), 10.5 (s, 1H, NH). ¹³C-NMR (DMSO-d6): δ 21.37, 112.09, 117.27, 126.11, 130.58, 135.35, 146.84, 150.04, 158.25, 159.0, 160.09, 165.33. MS analysis for C₁₃H₁₃N₃O: Calcd mass: 305.11, found (m/z, ES+): 306.

### Synthesis of 4-(pyrimidin-2-ylamino)-N-(1H-[1,2,4]-triazol-3-yl)-benzamide (RH7)

### Synthesis of 4-nitro-N-(1H-[1,2,4]-triazol-3-yl)-benzamide (21)

The 4-nitro benzoyl chloride (14, 10 mmol) in anhydrous chloroform was added dropwise to a cooled solution of 1H-[1,2,4]triazol-3-ylamine (20, 10 mmol) in 10 mL pyridine and stirred at 0° C. for 3 h. The formed precipitate was filtered and recrystallized from THE. Yield (70%). ¹H-NMR (DMSO-d6): δ 7.80 (s, 1H, NH), 8.06 (d, 1H, J=6.5, ArH), 8.21 (d, 2H, J=8.5, ArH), 8.35 (d, 2H, J=7.5, ArH), 10.5 (s, 1H, NH). MS analysis for C₉H₇N₅O₃: Calcd mass: 233.05, found (m/z, ES+): 234.

### Synthesis of 4-amino-N-(1H-[1,2,4]-triazol-3-yl)-benzamide (22)

The reduction of 4-nitro-N-(1H-[1,2,4]-triazol-3-yl)-benzamide (21, 10 mmol) to the corresponding amine was performed using Pd/C 10% in anhydrous THF (10 mL) and bubbled with hydrogen gas. The mixture was filtered through celite and evaporated to dryness for further purification. Yield (62%), ¹H-NMR (DMSO-d6): δ 7.67 (s, 2H, NH2), 7.84 (s, 1H, NH), 8.11 (d, 1H, J=7, ArH), 8.26 (d, 2H, J=7.5, ArH), 8.54 (d, 2H, J=7.5, ArH), 10.5 (s, 1H, NH). MS analysis for C₉H₉N₅O: Calcd mass: 203.08, found (m/z, ES+): 204.

### Synthesis of 4-(pyrimidin-2-ylamino)-N-(1H-[1,2,4]-triazol-3-yl)-benzamide (RH7)

A mixture of 4-amino-N-(1H-[1,2,4]-triazol-3-yl)-benzamide (22, 10 mmol) and 2-chloropyrimidine (8, 10 mmol) in 20 mL aqueous HCl was stirred for 18 h. The formed precipitate was filtered, washed with NaHCO₃, and purified with silica column chromatography using (8 chloroform: 2 methanol). Yield (69%). ¹H-NMR (D(MSO-d6): d 6.97 (m, 2H, ArH), 7.61 (s, 1H, NH), 7.63 (s, 1H, NH), 7.94 (d, 2H, J=8.5, ArH), 8.12 (d, 2H, J=8.7, ArH), 8.5 (d, 2H, J=6.5, ArH), 10.17 (s, 1H, NH). ¹³C-NMR (DMSO-d6): δ 106.33, 114.31, 117.57, 122.92, 127.89, 130.79, 141.52, 154.80, 159.66, 167.55. MS analysis for C₁₃H₁₁N₇O: Calcd mass: 281.10, found (m/z, ES+): 282.

### Synthesis of 4-tert-butoxycarbonylamino-benzoic acid 4-amino-6-o-tolylamino-[1.3.5]-triazin-2-ylmethyl ester (RH 8)

### Synthesis of 4-(tert-butoxycarbonyl)-amino-benzoic acid (23)

Para-amino benzoic acid (7, 4 mmol) was dissolved in 20 mL dioxane:water (1:1). The reaction mixture was stirred for 1 h at room temperature, then di-tert-butyl dicarbonate (23, 8 mmol) and TEA (8 mmol) was slowly added.

The reaction mixture was stirred at room temperature for 36 h. The reaction mixture was acidified with 3M HCl to pH 6. The precipitate was filtered and washed with cold water and dried. Yield 84%. mp 197-199° C. ¹H-NMR (DMSO-d6): δ 1.49 (s, 9H, tert-butyl), 7.45 (d, 2H, J=8, ArH), 7.83 (d, 2H, J=8.6, ArH), 9.72 (s, 1H, NH), 12.80 (s, 1H, COOH).

### Synthesis of 4-tert-butoxycarbonylamino-benzoic acid 4-amino-6-o-tolylamino-[1,3,5]triazin-2-ylmethyl ester (RH 8)

A mixture of 6-chloromethyl-N-o-tolyl-[1,3,5]-triazine-2,4-diamine (3, 5 mmol) and 4-(tert-butoxycarbonyl)-amino-benzoic acid (22, 10 mmol) in anhydrous THE (10 mL) and TBAF (20 mmol) was added dropwise. The reaction mixture was heated under reflux for 2 h and then evaporated under reduced pressure. Partition of the formed residue between 20 mL ethyl acetate and 10 mL water was performed followed by separation of the organic layer, which was then washed with saturated sodium bicarbonate (30 mL), dried over anhydrous sodium sulphate, and evaporated under reduced pressure. The purification of the obtained solid with column chromatography was performed with chloroform:methanol (20:1) as eluent. Yield 54%. ¹H-NMR (DMSO-d6): δ 1.48 (s, 9H, tert-butyl), 2.15 (s, 3H, CH3), 4.96 (s, 2H, CH2), 6.88 (s, 2H, NH2), 7.07 (d, 2H, J=6.7, ArH), 7.29 (m, 2H, ArH), 7.58 (d, 2H, J=8.75, ArH), 7.88 (d, 2H, J=8.45, ArH), 8.77 (s, 1H, NH), 9.79 (s, 1H, NH). ¹³C-NMR (DMSO-d6): δ 22.71, 28.11, 32.18, 69.85, 111.90, 118.16, 122.80, 125.81, 130.20, 132.09, 136.68, 144.33, 152.61, 164.95, 165.25, 166.83, 172.78, 206.76. MS analysis for C₂₃H₂₆N₆O₄: Calcd mass: 450.20, found (m/z, ES+): 451.19.

### Synthesis of 6-(2-amino-6-methyl-pyrimidin-4-yloxymethyl)-N-o-tolyl-[1,3,5]triazine-2,4-diamine (RH9)

A mixture of 6-chloromethyl-N-o-tolyl-[1,3,5]-triazine-2,4-diamine (3, 10 mmol), 2-amino-6-methyl-pyrimidin-4-ol (24, 10 mmol) and potassium carbonate (2 gm) in anhydrous dioxane (15 mL) was refluxed for 7 h. After cooling, the precipitate was collected and purified with crystallization from dioxane. Yield 62%. 1H-NMR (DMSO-d6): δ 2.0 (s, 3H, CH3), 2.18 (s, 3H, CH3), 3.42 (s, 2H, NH2), 4.28 (s, 2H, NH2), 5.48 (s, 2H, CH2), 6.89 (s, 1H, ArH), 7.04 (t, 1H, J=7.5, ArH), 7.10 (t, 1H, J=8, ArH), 7.17 (d, 1H, J=8.05, ArH), 7.31 (d, 1H, J=7.45, ArH), 8.71 (s, 1H, NH). MS analysis for C₁₆H₁₈N₈O: Calcd mass: 338.16, found (m/z, ES+): 339.

### Synthesis of N-pyrazin-2-yl-4-(pyrimidin-2-ylamino)-benzamide (RH10)

The 4-(pyrimidin-2-ylamino)-benzoic acid (9, 2 mmol) was dissolved in DMF and stirred under N2 at 0° C. for 10 min. TEA (8 mmol) and TBTU (4 mmol) were added and stirred for additional 30 min. 2-Amino pyrazine (25, 2.5 mmol) was added and stirred overnight. The product was partitioned between ethyl acetate and cold water, the organic layer was dried over anhydrous sodium sulphate and evaporated under reduced pressure. The solid obtained was purified with preparative TLC using ethyl acetate:petroleum ether (1:4) as eluent. Yield 52%. ¹H-NMR (DMSO-d6): δ 7.04 (t, 1H, J=5, ArH), 7.44-7.47 (m, 1H, ArH), 7.67 (d, 1H, J=7.5, ArH), 7.89 (d, 2H, J=7, ArH), 8.11-8.14 (m, 2H, ArH), 8.18 (s, 1H, NH), 8.27 (d, 1H, J=7.5, ArH), 8.64 (d, 2H, J=5.5, ArH), 10.4 (s, 1H, NH). ¹³C-NMR (DMSO-d6): δ 109.29, 113.38, 118.31, 122.90, 124.53, 127.04, 131.51, 144.77, 156.00, 158.05, 159.64, 169.00. MS analysis for C₁₅H₁₂N₆O: Calcd mass: 292.11, found (m/z, ES+): 292.9.

### Synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide (RH11)

### (2R,3R,4S,5R)-2-(4-Amino-pyrrolo[2,1-f]-[1,2,4]-triazin-7-yl)-3,4-dihydroxy-5-hydroxymethyl-tetrahydro-furan-2-carbonitrile (GS-441524)

¹H-NMR (DMSO-d6): δ 3.29 (s, 1H, CH), 3.49-3.51 (m, 1H, CH), 3.64-3.66 (m, 1H, CH), 3.94-3.98 (m, 1H, CH), 4.04-4.07 (m, 1H, CH), 4.64 (t, 1H, J=6.5, OH), 4.91 (t, 1H, J=6, OH), 5.20 (d, 1H, J=4, OH), 6.08 (d, 1H, J=6.5, ArH), 6.87 (d, 1H, J=5, ArH), 6.90 (d, 1H, J=5, ArH), 7.80 (s, 2H, NH2). MS analysis for C₁₂H₁₃N₅O₄: Calcd mass: 291.10, found (m/z, ES+): 292.

### Synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide (RH11)

GS-441524 (7, 2 mmol) was dissolved in 10 mL anhydrous pyridine under inert condition. EDCI (6 mmol), 4-guanidino-benzoic acid (26, 2 mmol) and DMAP (0.2 mmol) were added sequentially and stirred at room temperature overnight and concentrated under vacuum for further purification with column chromatography using gradient elution with methanol:chloroform (7:3). ¹H-NMR (DMSO-d6): δ 3.22 (s, 1H, CH), 3.47-3.52 (m, 1H, CH), 3.62-3.66 (m, 1H, CH), 3.94-3.98 (m, 1H, CH), 4.04-4.06 (m, 1H, CH), 4.64 (t, 1H, J=6.5, OH), 4.91 (t, 1H, J=6, OH), 5.19 (d, 1H, J=4, OH), 5.97 (m, 3H, J=4, 2CH, ArH), 6.02 (m, 3H, J=6.5, ArH), 6.09 (d, 1H, J=5.5, ArH), 6.88 (d, 2H, J=5.5, ArH), 7.69 (s, 1H, NH), 7.90 (s, 1H, NH), 9.87 (s, 1H, NH). ¹³C-NMR (DMSO-d6): δ 63.03, 74.26, 78.95, 83.70, 86.03, 101.69, 109.08, 110.69, 116.69, 117.58, 123.33, 131.65, 142.34, 147.89, 152.77, 153.95, 155.51, 164.26. MS analysis for C₂₀H₂₀N₈O₅: Calcd mass: 452.16, found (m/z, ES+): 452.99.

### Synthesis of 4-guanidino-benzoic acid 4-isopropyl-2-oxo-2H-chromen-6-yl ester (RH12)

The 4-guanidino-benzoic acid (26, 2 mmol) was dissolved in 10 mL anhydrous pyridine under inert condition. EDCI (6 mmol), 7-hydroxy-4-isopropylcoumarin (6, 2 mmol) and DMAP (0.2 mmol) were added sequentially and stirred at room temperature overnight and concentrated under vacuum for further purification with column chromatography using gradient elution with methanol:ethyl acetate:water:acetic acid (5:5:1:1). ¹H-NMR (DMSO-d6): δ 1.29 (d, 6H, 2 CH3 of isopropyl), 3.43-3.45 (m, 1H, CH of isopropyl), 6.35 (s, 1H, ArH), 7.35 (dd, 1H, J=8.5, ArH), 7.43-7.48 (m, 3H, ArH), 7.76 (s, 3H, NH), 7.97 (d, 1H, J=8.5, ArH), 8.18 (d, 2H, J=7.5, ArH), 10.12 (s, 1H, NH). ¹³C-NMR (DMSO-d6): δ 21.50, 28.14, 109.08, 110.69, 115.69, 118.58, 123.33, 124.84, 126.01, 131.65, 142.34, 152.77, 153.95, 155.51, 160.45, 162.24, 164.26. MS analysis for C₂₀H₁₉N₃O₄: Calcd mass: 365.95, found (m/z, ES+): 366.14.

### Synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide (RH13)

### Synthesis of (2R,3R,4S,5R)-2-(4-amino-pyrrolo[2,1-f]-[1,2,4]-triazin-7-yl)-3,4-bis (tert-butyll-dimethylsilyl)-dihydroxy-5-(tert-butyll-dimethylsilyl)-hydroxymethyl-tetrahydro-furan-2-carbonitrile (27)

A solution of GS-441524 (3 mmol) in DMF (10 ml), imidazole (30 mmol), DMAP (3 mmol) and TBDMSCI (15 mmol) were mixed under inert condition and the mixture was stirred for 8 h. The reaction was quenched with ethyl acetate:water (1:3) and washed with aqueous NaHCO₃. The organic layer was dried over sodium sulfate anhydrous and evaporated over reduced pressure to afford yellow solid that was purified with silica gel column chromatography using (20 DCM: 1 methanol). ¹H-NMR (DMSO-d6): δ 0.11 (s, 6H, 2CH3), 0.14 (s, 6H, 2CH3), 0.16 (s, 6H, 2CH3), 0.20 (s, 6H, 2CH3), 0.73 (s, 9H, tert-butyl), 0.84 (s, 9H, tert-butyl), 0.95 (s, 9H, tert-butyl), 3.49-3.51 (m, 1H, CH), 3.64-3.66 (m, 1H, CH), 3.94-3.98 (m, 1H, CH), 4.04-4.07 (m, 1H, CH), 5.02 (d, 1H, J=4.5, CH), 6.08 (d, 1H, J=6.5, ArH), 6.87 (d, 1H, J=5, ArH), 6.90 (d, 1H, J=5, ArH), 7.80 (s, 2H, NH2). MS analysis for C₃₀H₅₅N₅O₄Si₃: Calcd mass: 633.36, found (m/z, ES+):634.12.

### Synthesis of (2R,3R,4S,5R)-N-{7-[3,4-bis-(tert-butyl-dimethyl-silanyloxy)-5-(tert-butyl-dimethyl-silanyloxymethyl)-2-cyano-tetrahydro-furan-2-yl]-pyrrolo[2,1-f]-[1,2,4]-triazin-4-yl-4-(pyrimidin-2-ylamino)-benzamide (28)

The 4-(pyrimidin-2-ylamino)-benzoic acid (9, 2 mmol) was dissolved in DMF and stirred under N2 at 0° C. for 10 min. TEA (8 mmol) and TBTU (4 mmol) were added and stirred for additional 30 min. (2R,3R,4S,5R)-2-(4-amino-pyrrolo[2,1-f]-[1,2,4]-triazin-7-yl)-3,4-bis (tert-butyl-dimethylsilyl)-dihydroxy-5-(tert-butyl-dimethylsilyl)-hydroxymethyl-tetrahydro-furan-2-carbonitrile (27, 2.5 mmol) was added and stirred overnight. Iced water was added, and the product was extracted with ethyl acetate. The organic layer was dried over anhydrous sodium sulphate and evaporated under reduced pressure. The solid obtained was purified with column chromatography using gradient methanol:chloroform as an eluent. MS analysis for C₄₁H₆₂N₈O₅Si₃: Calcd mass: 830.40, found (m/z, ES+): 831.

### Synthesis of (2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide (RH13)

Compound (29, 2 mmol) was dissolved in anhydrous THE (10 mL). TBAF (1M in THF) was added, and the reaction was left overnight with stirring at room temperature. The reaction was concentrated under reduced pressure and the residue was purified with column chromatography using gradient (methanol:chloroform) eluent. Yield 43%. ¹H-NMR (DMSO-d6): δ 3.27- (d, 1H, J=4, OH), 3.71-3.74 (m, 1H, CH), 3.85-3.88 (m, 1H, CH), 3.96-3.98 (m, 1H, CH), 4.03-4.05 (m, 1H, CH), 4.28-4.30 (m, 1H, CH), 4.73 (t, 1H, J=5.5, OH), 5.20 (d, 1H, J=4, OH), 5.80 (s, 1H, NH), 6.08 (d, 1H, J=5.65, ArH), 6.27 (d, 1H, J=6, ArH), 6.51 (t, 1H, J=5, ArH), 6.80 (d, 2H, J=7.5, ArH), 7.79 (d, 2H, J=8, ArH), 8.29 (d, 2H, J=5.5, ArH), 9.95 (s, 2H, NH2). ¹³C-NMR (DMSO-d6): δ 60.91, 66.42, 69.85, 77.53, 85.27, 99.51, 102.27, 111.26, 116.87, 117.31, 122.99, 125.23, 129.64, 132.25, 146.93, 154.19, 155.72, 160.95, 163.20. MS analysis for C₂₃H₂₀N₈O₅: Calcd mass: 488.16, found (m/z, ES+): 489.

### Synthesis of 4-guanidino-N-(9H-purin-6-yl)-benzamide (RH14)

The 4-guanidino-benzoic acid (26, 2 mmol) was dissolved in 10 mL anhydrous pyridine under inert condition. EDCI (6 mmol), adenine (13, 3 mmol) and DMAP (0.2 mmol) were added sequentially and stirred at 50° C. overnight. The product was then concentrated under vacuum for further purification with column chromatography using gradient elution with methanol:chloroform. Yield 50%, ¹H-NMR (DMSO-d6): δ 4.5 (s, 2H, NH2) 8.24 (d, 2H, J=7.5, ArH), 8.36 (d, 2H, J=7, ArH), 8.49-8.51 (m, 2H, ArH), 9.07 (s, 1H, NH), 9.5 (s, 1H, NH), 11.5 (s, 1H, NH), 12.22 (s, 1H, NH). MS analysis for C₁₃H₁₂N₈O: Calcd mass: 296.12, found (m/z, ES+): 297.

### Synthesis of N-(5-fluoro-2-oxo-1,2-dihydro-pyrimidin-4-yl)-4-guanidino-benzamide (RH15)

The 4-guanidino-benzoic acid (26, 2 mmol) was dissolved in 10 mL anhydrous pyridine under inert condition. EDCI (6 mmol), DMAP (0.2 mmol) and 4-amino-5-fluoro-1H-pyrimidin-2-one (29, 3 mmol) were added sequentially and stirred at room temperature overnight and concentrated under vacuum for further purification with column chromatography using gradient elution with methanol:chloroform:acetic acid (3:3:1). Yield 60%, ¹H-NMR (DMSO-d6): δ 3.4 (s, 2H, NH2), 6.87 (t, 1H, J=5.5, ArH), 7.28 (d, 2H, J=8.3, ArH), 7.81 (d, 2H, J=8.6, ArH), 8.50 (s, 1H, NH), 8.98 (s, 1H, NH), 9.82 (s, 1H, NH), 10.42 (s, 1H, NH). ¹³C-NMR (DMSO-d6): δ 101.25, 114.23, 123.74, 125.45, 128.30, 148.63, 158.30, 160.78, 164.43, 167.00. MS analysis for C₁₂H₁₁FN₆O₂: Calcd mass: 290.09, found (m/z, ES+): 291.

### Synthesis of N-[1-(3,4-dihydroxy-5-hydroxymethyl-tetrahydro-furan-2-yl)-4-oxo-1,4-dihydro-pyrimidin-2-yl]-4-guanidino-benzamide (RH16)

The 4-guanidino-benzoic acid (26, 3 mmol) was dissolved in 10 mL anhydrous pyridine under inert condition. EDCI (6 mmol), DMAP (0.3 mmol) and 4-amino-1-[(2R,3R,4S,5R)-3,4-dihydroxy-5-(hydroxymethyl)-oxolan-2-yl]pyrimidin-2-one (30, 3 mmol) were added sequentially and stirred at room temperature overnight and then concentrated under vacuum for further purification with column chromatography using gradient elution with methanol:chloroform:water:acetic acid (3:3:1:1). Yield 40%. ¹H-NMR (DMSO-d6): δ 2.10 (s, 1H, NH), 3.49-3.52 (m, 1H, CH), 3.64-3.67 (m, 1H, CH), 3.69-3.72 (m, 1H, CH), 3.97-3.99 (m, 1H, CH), 4.30-4.34 (m, 1H, CH), 4.67 (d, 1H, J=5.5, OH), 5.09 (s, 1H, NH), 5.18 (d, 1H, J=4.5, OH), 5.42 (t, 1H, J=5.5, OH), 5.53 (d, 1H, J=5.5, CH), 6.35 (d, 1H, J=7.5, ArH), 6.41 (d, 1H, J=6.5, ArH), 6.76 (s, 2H, NH2), 7.48 (d, 2H, J=7.8, ArH), 7.73 (d, 2H, J=7.5, ArH), 10.08 (s, 1H, NH). ¹³C-NMR (DMSO-d6): δ 63.70, 69.16, 73.84, 79.34, 84.73, 101.60, 115.20, 125.17, 131.90, 142.80, 148.16, 150.07, 161.09, 165.10. MS analysis for C₁₇H₂₀N₆O₆: Calcd mass: 404.14, found (m/z, ES+): 404.9.

### Synthesis of N-(5-fluoro-2-oxo-1,2-dihydro-pyrimidin-4-yl)-4-(pyrimidin-2-ylamino)-benzamide (RH17)

The 4-(pyrimidin-2-ylamino)-benzoic acid (9, 10 mmol) was dissolved in 10 mL anhydrous pyridine under inert condition. EDCI (6 mmol), DMAP (0.2 mmol) and 4-amino-5-fluoro-1H-pyrimidin-2-one (29, 3 mmol) were added sequentially and stirred at room temperature overnight and concentrated under vacuum for further purification with column chromatography using gradient elution with methanol:chloroform (1:4). Yield 45%, ¹H-NMR (DMSO-d6): δ 4.2 (s, 1H, NH), 6.80 (t, 1H, J=5, ArH), 7.30 (d, 2H, J=8.65, ArH), 7.79 (d, 2H, J=8.3, ArH), 8.60 (m, 1H, ArH), 8.76 (d, 2H, J=6.5, ArH), 9.80 (s, 1H, NH), 10.42 (s, 1H, NH). ¹³C-NMR (DMSO-d6): δ 101.25, 114.23, 123.74, 125.45, 128.30, 148.63, 158.30, 160.78, 164.43, 167.00. MS analysis for C₁₅H₁₁FN₆O₂: Calcd mass: 326.09, found (m/z, ES+): 327.

### Synthesis of 4-guanidino-N-(2-oxo-1,2-dihydro-pyrimidin-4-yl)-benzamide (RH18)

The 4-guanidino-benzoic acid (26, 3 mmol) was dissolved in 10 mL anhydrous pyridine under inert condition. EDCI (6 mmol), DMAP (0.3 mmol) and 6-amino-1H pyrimidin-2-one (10, 3 mmol) were added sequentially and stirred at room temperature overnight and concentrated under vacuum for further purification with column chromatography using gradient elution with methanol:chloroform:water:acetic acid (3:6:1:1). Yield 42%. ¹H-NMR (DMSO-d6): δ 4.24 (s, 2H, NH2), 6.50 (d, 1H, J=5.5, ArH), 6.74 (d, 1H, J=4.5, ArH), 7.58 (d, 2H, J=7.5, ArH), 7.78 (d, 2H, J=8, ArH), 8.80 (s, 1H, NH). 9.35 (s, 1H, NH), 9.73 (s, 1H, NH) 10.28 (s, 1H, NH). ¹³C-NMR (DMSO-d6): δ 102.40, 113.20, 115.17, 125.70, 131.90, 141.80, 147.16, 207, 160.09, 164.10. MS analysis for C₁₂H₁₂N₆O₂: Calcd mass: 272.10, found (m/z, ES+): 273.

### Synthesis of N-[1-(3,4-dihydroxy-5-hydroxymethyl-tetrahydro-furan-2-yl)-2-oxo-1,2-dihydro-pyrimidin-4-yl]-4-(pyrimidin-2-ylamino)-benzamide (RH19)

The 4-(pyrimidin-2-ylamino)-benzoic acid (9, 10 mmol) was dissolved in 10 mL anhydrous pyridine under inert condition. EDCI (6 mmol), DMAP (0.2 mmol) and 4-amino-1-[(2R,3R,4S,5R)-3,4-dihydroxy-5-(hydroxymethyl)-oxolan-2-yl] pyrimidin-2-one (30, 3 mmol) were added sequentially and stirred at room temperature overnight and concentrated under vacuum for further purification with column chromatography using gradient elution with methanol:chloroform (3:7). Yield 49%, ¹H-NMR (DMSO-d6): δ 3.58-3.63 (m, 1H, CH), 3.73-3.77 (m, 1H, CH), 3.90-3.92 (m, 1H, CH), 3.97-4.03 (m, 2H, CH), 5.07 (d, 1H, J=6, OH), 5.20 (t, 1H, J=5.5, OH), 5.50 (d, 1H, J=6.4 OH), 5.81 (d, 1H, J=4, CH), 6.95 (t, 1H, J=4.9, ArH), 7.34 (s, 1H, NH), 7.90 (d, 3H, J=8.5, ArH), 7.99 (d, 2H, J=8.3, ArH), 8.46 (d, 1H, J=7.4, ArH), 8.56 (d, 2H, J=4.8, ArH), 10.08 (s, 1H, NH). ¹³C-NMR (DMSO-d6): δ 60.18, 66.40, 68.96, 69.83, 75.70, 94.89, 112.09, 116.87, 123.54, 128.67, 142.81, 145.57, 155.79, 159.60, 164.34, 167.80. MS analysis for C₂₀H₂₀N₆O₆: Calcd mass: 440.14, found (m/z, ES+): 441.

### Synthesis of (2R,3R,4S,5R)-{4-[7-(2-cyano-3,4-dihydroxy-5-hydroxymethyl-tetrahydro-furan-2-yl)-pyrrolo[2,1-f]-[1,2,4]-triazin-4-ylcarbamoyl]-phenyl}-carbamic acid tert-butyl ester (RH20)

The GS-441524 (3 mmol) was dissolved in DMF and stirred under N₂ at 0° C. for 10 min. TEA (8 mmol) and TBTU (4 mmol) was added and stirred for additional 30 min. 4-(Tert-butoxycarbonyl)-amino-benzoic acid (25, 2.5 mmol) was added and stirred overnight. The product was partitioned between ethyl acetate and cold water, the organic layer was dried over anhydrous sodium sulphate and evaporate under reduced pressure. The solid obtained was purified with preparative TLC using ethyl acetate:petroleum ether (1:5) as eluent. Yield 50%. ¹H-NMR (DMSO-d6): δ 1.5 (s, 9H, tert-butyl), 3.45-3.47 (m, 1H, CH), 3.71-3.74 (m, 1H, CH), 3.84-3.88 (m, 1H, CH), 3.96-3.99 (m, 1H, CH), 4.03-4.05 (m, 1H, CH), 4.29 (d, 1H, J=4.5, OH), 4.73 (t, 1H, J=6.5, OH), 4.90 (t, 1H, J=4.5, OH), 5.98 (d, 1H, J=4.5, ArH), 6.08 (d, 1H, J=5.5, ArH), 6.26 (d, 1H, J=5.5, ArH), 7.42 (d, 2H, J=8, ArH), 7.94 (d, 2H, J=7.5, ArH), 9.25 (s, 1H, NH), 9.95 (s, 1H, NH). ¹³C-NMR (DMSO-d6): δ 28.30, 32.12, 59.52, 60.25, 79.56, 81.40, 84.03, 108.81, 114.78, 117.56, 118.98, 125.14, 129.08, 130.01, 133.89, 143.90, 159.64, 164.25. MS analysis for C₂₄H₂₆N₆O₇: Calcd mass: 510.19, found (m/z, ES+): 511.04.

### Synthesis of 4-guanidino-N-pyrazin-2-yl-benzamide (RH21)

### Synthesis of 4-nitro-N-pyrazin-2-yl-benzamide (31)

The 4-nitro benzoyl chloride (14, 10 mmol) in anhydrous chloroform was added dropwise to a cooled solution of 2-amino pyrazine (25, 10 mmol) in 10 mL pyridine and stirred at room temperature for 4 h. The formed precipitate was filtered and recrystallized from methanol. Yield (64%). ¹H-NMR (DMSO-d6): δ 8.24 (d, 2H, J=8.5, ArH), 8.36 (d, 2H, J=8, ArH), 8.46 (d, 1H, J=2.5, ArH), 8.5-8.52 (m, 1H, ArH), 9.42 (d, 1H, J=2, ArH) 9.2 (s, 1H, NH). MS analysis for C₁₁H₈N₄O₃: Calcd mass: 244.06, found (m/z, ES+): 244.99.

### Synthesis of 4-amino-N-pyrazin-2-yl-benzamide (32)

The 4-nitro-N-pyrazin-2-yl-benzamide (31) was reduced to the corresponding amine using Pd/C 10% in anhydrous THE (10 mL) and bubbled with hydrogen gas. The mixture was filtered through celite and evaporated to dryness for further purification. ¹H-NMR (DMSO-d6): δ 6.02 (s, 2H, NH2), 8.24 (d, 2H, J=7.5, ArH), 8.41 (d, 1H, J=2.5, ArH), 8.48 (d, 2H, J=7, ArH), 8.9 (d, 1H, J=4.5, ArH), 9.42 (d, 1H, J=2, ArH), 11.5 (s, 1H, NH). MS analysis for C₁₁H₁₀N₄O: Calcd mass: 214.09, found (m/z, ES+): 215.

### Synthesis of 4-guanidino-N-pyrazin-2-yl-benzamide (RH21)

The cyanamide (10 mmol) and nitric acid solution (7 mmol) was added to a solution of 4-amino-N-pyrazin-2-yl-benzamide (32, 7 mmol) in aqueous ethanol 20 mL. The reaction mixture was heated under reflux for overnight. Diethyl ether (20 mL) was added and the solid obtained was collected and purified with column chromatography using butanol:water:ethyl acetate (1:1:4). ¹H-NMR (DMSO-d6): δ 4.2 (s, 2H, NH2), 7.03 (d, 2H, J=7.5, ArH), 7.70 (s, 1H, NH), 7.99 (d, 2H, J=7.5, ArH), 7.70 (s, 2H, NH2), 8.38 (d, 1H, J=2.5, ArH), 8.45 (d, 1H, J=4, ArH), 9.5 (s, 1H, NH). ¹³C-NMR (DMSO-d6): d 121.15, 128.60, 130.10, 135.09, 139.74, 141.13, 143.15, 148.84, 164.31, 167.60. MS analysis for C₁₂H₁₂N₆O: Calcd mass: 256.11, found (m/z, ES+): 257.

### Synthesis of (2R,3R,4S,5R)-{N-[7-(2-cyano-3,4-dihydroxy-5-hydroxymethyl-tetrahydro-furan-2-yl)-pyrrolo[2,1-f-]-[1,2,4]-triazin-4-yl]-guanidine (RH22)

The cyanamide (10 mmol) and nitric acid solution (7 mmol) was added to a solution of GS-441524 (7 mmol) in aqueous ethanol (20 mL). The reaction mixture was stirred overnight at room temperature. Diethyl ether (20 mL) was added and the solid obtained was collected and purified with preparative TLC using butanol:water:ethyl acetate (1:1:4). ¹H-NMR (DMSO-d6): δ 2.1 (s, 2H, NH₂), 3.49-3.51 (m, 1H, CH), 3.64-3.66 (m, 1H, CH), 3.94-3.98 (m, 1H, CH), 4.04-4.07 (m, 1H, CH), 4.64 (t, 1H, J=6.5, OH), 4.91 (t, 1H, J=6, OH), 5.20 (d, 1H, J=4, OH), 5.8 (s, 1H, NH), 6.08 (d, 1H, J=6.5, ArH), 6.87 (d, 1H, J=5, ArH), 6.90 (d, 1H, J=5, ArH), 7.80 (s, 1H, NH). ¹³C-NMR (DMSO-d6): δ 60.95, 70.88, 74.25, 78.56, 85.42, 100.81, 110.78, 116.54, 117.36, 123.89, 147.90, 155.64. MS analysis for C₁₃H₁₅N₇O₄: Calcd mass: 333.10, found (m/z, ES+): 334.

### Synthesis of N-[1-(3,4-dihydroxy-5-hydroxymethyl-tetrahydro-furan-2-yl)-2-oxo-1,2-dihydro-pyrimidin-4-yl]-4-guanidino-benzamide (RH23)

The 4-guanidino-benzoic acid (26, 3 mmol) was dissolved in 10 mL anhydrous pyridine under inert condition. EDCI (6 mmol), DMAP (0.3 mmol) and 4-amino-1-[(2R,3R,4S,5R)-3,4-dihydroxy-5-(hydroxymethyl)oxolan-2-yl]-1,3,5-triazin-2-one (33, 3 mmol) were added sequentially and stirred at room temperature overnight and concentrated under vacuum for further purification with preparative TLC using acetic acid:water:ethyl acetate (1:1:4). ¹H-NMR (DMSO-d6): δ 3.43-3.53 (m, 1H, CH), 3.73-3.75 (m, 1H, CH), 3.90-3.92 (m, 1H, CH), 4.35-4.38 (m, 1H, CH), 4.79 (d, 1H, J=4.9, CH), 5.03 (t, 1H, J=5, OH), 5.18 (d, 1H, J=4.5, OH), 5.37 (d, 1H, J=6, OH), 5.81 (d, 1H, J=6, CH), 6.80 (s, 1H, NH), 6.90 (s, 2H, NH₂), 7.34 (d, 1H, J=8.5, ArH), 7.48 (d, 2H, J=7.5, ArH), 7.52 (d, 1H, J=7.5, ArH), 7.85 (d, 2H, J=8, ArH), 8.85 (s, 1H, NH), 10.08 (s, 1H, NH). ¹³C-NMR (DMSO-d6): 65.40, 68.06, 72.84, 75.77, 83.83, 102.60, 112.70, 128.67, 132.51, 144.80, 158.27, 159.00, 164.80. MS analysis for C₁₇H₂₀NO₆: Calcd mass: 404.14, found (m/z, ES+): 405.

### EXPERIMENTAL EXAMPLES

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### In Vitro Anti-SARS-CoV-2 Activity of RH 1-23 Compounds

The antiviral activity was carried out as previously described [29, 30]. Briefly, 96-well tissue culture plates were seeded with 2.4×104 Vero-E6 cells/and incubated overnight in Dulbecco's Modified Eagle's medium (DMEM) containing 10% Fetal Bovine Serum (FBS) and 1% penicillin/streptomycin antibiotic mixture at a humidified 37° C. incubator under 5% CO₂. The cells were treated with SARS-CoV-2 (NRC-03-nhCoV strain, accession #EPI_ISL_430820) for viral adsorption, which was further overlaid with 50 μl DMEM containing varying concentrations of the tested compounds. Following incubation at 37° C. and 5% C₀₂ for 72 h, the cells were fixed with 100 μl of 4% paraformaldehyde for 20 min and stained at room temperature with 0.1% crystal violet in distilled water for 15 min. The crystal violet dye was then dissolved in 100 μl methanol and the optical density of the obtained color was measured at 570 nm using Anthos Zenyth 200 rt plate reader (Anthos Labtec Instruments, Heerhugowaard, Netherlands). The IC₅₀ of the compound was measured using the below formula: =[(OD test−OD blank)÷(OD negative control−OD blank)]×100.

### TMPRSS2 Fluorogenic Assay

TMPRSS2 fluorogenic assay kit (CAT #78083, BPS Bioscience, San Diego, CA, USA) was used to test the TMPRSS2 inhibitory activity of the tested compounds according to reported method [30]. Briefly, 30 μl TMPRSS2 (5 ng/μl) was added to 10 μl of the tested compounds at different concentrations (0.001, 0.390, 0.750, 1.25, 2.5, 5, and 10 and μg/mL). Following incubation in dark for 45 min at room temperature, 10 μl TMPRSS2 substrate (50 μM) was added, and the fluorescence intensity was measured after 10 min incubation at room temperature with a microtiter plate-reader (Synergy H₁, Biotek Ltd, Winooski, VT, USA) at an excitation and emission wavelengths 383 and 455 nm, respectively. 10 μl Camostat mesylate (10 μM) was used as a positive control [31], while the reaction without inhibitor and enzyme was used as blank control.

### 2.3. SARS-CoV-2 RNA-Dependent RNA Polymerase (RdRp) In Vitro Inhibition Assay

In vitro SARS-CoV-2 RdRp inhibition activity was tested using viral RNA-dependent RNA polymerase assay kit (Cat #S2RPA100KE, Profoldin, Hudson, MA) according to the supplier protocol [32]. Briefly, 1 μl of 50× recombinant RdRp was incubated with 5 μl of 50× buffer and 42 μl of tested compounds at different concentrations (0, 10, 25, 50, and 100 μg/ml) in water at room temperature for 15 min. The reaction was initiated by the addition of master mix of 1 μl of 50× template (as a single-stranded polyribonucleotide) and 1 μl of 50×NTPs. The reaction (50 μl) was incubated for 2 h at 37° C. and later stopped by the addition of 65 μl of 1× fluorescence dye. The fluorescence signal was recorded with a fluorescence spectrometer (Synergy H1, Biotek Ltd) within 10 min using excitation and emission filters at 488 and 535 nm, respectively. For positive and negative controls, reactions were employed with remdesivir (50 μg/ml) and water instead of inhibitors, respectively. The results were expressed as a % of the negative control.

### Main Protease (M^{pro}) Assay

M pro assay was carried out using 3CL Protease Untagged (SARS-CoV-2) Kit Assay (CAT #78042-1, BPS Bioscience, USA) [30]. 10 μl of 25 μg/mL of the tested compounds were incubated with 30 μl M^{pro} enzyme (15 ng per reaction) in 96 black flat-bottom well plate. The reaction was performed in a reaction buffer with 1 mM 1,4-dithio-D, L-threitol (DTT) and the incubation was performed for 45 min at room temperature with slow shaking. Following incubation, 10 μl of 80 mM M^{pro} substrate was added in dark and allowed to incubate for 60 min at room temperature. The fluorescence intensity was measured in a microtiter plate-reading fluorimeter (Synergy H1, Biotek Ltd, Winoosk, VT, USA) at an excitation and emission wavelengths 360 and 460 nm, respectively. The inhibition activity of the compound was screened at concentrations 25 μg/mL, while GC376 was employed as positive control at concentration 100 μM [33], and the reaction without inhibitor and enzymes was employed as blank.

### Mechanism of Action

### Adsorption Mechanism

The viral adsorption mode of action was tested according to reported method with minor modifications [34]. Briefly, Vero E6 cells were seeded in a 6-well plate and incubated for 24 h at 37° C. The cells were washed with PBS and then treated with 10 μg/ml of the tested compounds. This was followed by incubation for 1 h. The virus in 3 ml DMEM with 2% agarose mixture was added after washing of the non-adsorbed material with PBS and incubated for 1 h. The solidified plates were incubated until the formation of viral plaques at 37° C. for 4 days. Untreated cells were employed as negative control. The plaques were fixed using 10% formalin solution for 1 h and stained using crystal violet.

### Replication Mechanism

The inhibition activity of the tested compounds on viral replication was studied according to previously reported protocol with minor modifications [35]. Briefly, Vero E6 cells were seeded in 6-well plate and incubated at 37° C. for 24 h. The cells were infected with the virus and incubated for 1 h at 37° C. The cells were washed using PBS to remove non-adsorbed virus. The infected cells were treated with 10 μg/ml of the tested compounds and then incubated for 1 h, and 3 ml of DMEM supplemented with 2% agarose mixture was added. Plates were incubated for three days then fixed using 10% formalin solution for 1 h and stained with crystal violet. Uninfected cells were employed as negative control.

### Virucidal Mechanism

The virucidal activity of the compounds was studied according to previously adapted method [36]. Briefly, Vero E6 cells were seeded in 6-well plate and incubated at 37° C. for 24 h. The cells were infected with 120 μL of the virus.

The infected cells were treated with 10 μg/ml concentration of the tested compounds and incubated for 1 h at room temperature. Uninfected control cells were employed as negative control. The mixture was added to the Vero E6 cell in DMEM medium and then incubated for three days.

### Statistical Analysis

The data were obtained and graphed using GraphPad Prism (5.0, GraphPad Inc., La Jolla, CA, USA). The enzyme inhibition activities of the compounds against RdRp, TMPRSS2 and M^{pro} were analyzed by one-way analysis of variance (ANOVA) using Dunnet's multiple comparisons test. P<0.05 was considered as significant. The data display the mean±_SEM of at least 3 replicas.

### Computational Studies

All computational work was carried out using Schrödinger suite 12.7 available at www.Schrödinger.com and using Maestro graphical user interface software.

### Protein Preparation

The 3D crystal structures of SARS-CoV-2 RdRp (PDB ID: 7BV2) and TMPRSS2 (PDB ID: 20Q5) enzymes were downloaded from the protein data bank (https://www.rcsb.org/). The proteins were prepared and refined using the Protein Preparation Wizard [37]. Crystallographic water molecules that beyond 5 Å were removed. All the missing hydrogen atoms were added at pH 7.3 for appropriate ionization and the tautomerization state of amino acid residues and proper bond order were assigned. Next, the refining of protein structures was performed and the water molecules with <3 hydrogen bonds to non-waters were deleted. Finally, the energy minimization was done using OPLS-4 to relieve the steric clashes [38].

### Ligand Preparation

The 2D structures of the generated library were converted to 3D structures using LigPrep, Schrodinger [39]. Hydrogen atoms were added, and the salt ions were removed. The most probable ionization states were calculated at pH 7.3 using the Epik module [40, 41]. During the ligand preparation, specified chirality of the 3D crystal structure was retained. The subsequent energy minimization of each structure was carried out using OPLS4 force field [38] and filtered through a relative energy tool to exclude the high energy structures from the given input. Besides, any errors in the ligands were eradicated in order to enhance the accuracy of the molecular docking [42].

### Grid Generation

The ligand in crystal structure of RdRp and TMPRSS2 enzymes were used for grid generation. A grid box was generated at the centroid of the active site for docking studies and the active site was defined around the ligand crystal structure.

### Molecular Docking

Molecular docking was performed within the catalytic pocket site of the proteins using standard precision (SP) mode of Grid using Glide [43, 44]. The prepared ligands were docked against grid generated RdRP (PDB: 7BV2) and TMPRSS2 (PDB ID: 20Q5) in SP flexible mode [45].

### Physiochemical, Pharmacokinetic, and Drug-Likeness Properties of the Potent Candidates

The identified potential compounds will be subjected to in silico ADMET predictions to select the ones that can exhibit excellent ADMET (absorption, distribution, metabolism, elimination, toxicity). Good bioavailability can be achieved with an appropriate balance between solubility and partitioning properties. In addition, total polar surface area (TPSA) and number of rotatable bonds are good descriptors for the absorption and bioavailability. Molecular properties (TPSA, Log P, OH—NH interaction, number of rotating bands, drug-likeness, potential toxicity, and molecular weight) of the new synthesized compounds were calculated using swiss-ADME server. ADME properties of the synthesized compounds were predicted and compared to remdesivir [46]. The compliance of the newly synthesized compounds to Lipinski's rule of five were evaluated for the discovery and development of novel drug candidate.

### REFERENCES

[1] Hamdy, R., et al., Iterated virtual screening-assisted antiviral and enzyme inhibition assays reveal the discovery of novel promising anti-sars-cov-2 with dual activity. International journal of molecular sciences, 2021. 22(16): p. 9057.
[2] Elseginy, S. A., et al., Promising anti-SARS-CoV-2 drugs by effective dual targeting against the viral and host proteases. Bioorganic & Medicinal Chemistry Letters, 2021. 43: p. 128099.
[3] Rosenberg, E. S., et al., New COVID-19 cases and hospitalizations among adults, by vaccination status-New York, May 3-Jul. 25, 2021. Morbidity and Mortality Weekly Report, 2021. 70(34): p. 1150.
[4] Encinar, J. A. and J. A. Menendez, Potential drugs targeting early innate immune evasion of SARS-coronavirus 2 via 2′-O-methylation of viral RNA. Viruses, 2020. 12(5): p. 525.
[5] Matthieu, S., et al., I (nsp1) ecting SARS-CoV-2-ribosome interactions. Communications Biology, 2021. 4(1).
[6] Tegally, H., et al., Emergence and rapid spread of a new severe acute respiratory syndrome-related coronavirus 2 (SARS-CoV-2) lineage with multiple spike mutations in South Africa. MedRxiv, 2020.
[7] Edara, V.-V., et al., Infection and vaccine-induced neutralizing-antibody responses to the SARS-CoV-2 B. 1.617 variants. New England Journal of Medicine, 2021. 385(7): p. 664-666.
[8] Dejnirattisai, W., et al., SARS-CoV-2 Omicron-B. 1.1. 529 leads to widespread escape from neutralizing antibody responses. Cell, 2022.
[9] Takashita, E., et al., Efficacy of Antibodies and Antiviral Drugs against Covid-19 Omicron Variant. New England Journal of Medicine, 2022.
[10] Belayneh, A., Off-label use of chloroquine and hydroxychloroquine for COVID-19 treatment in Africa against WHO recommendation. Research and Reports in Tropical Medicine, 2020. 11: p. 61.
[11] Eastman, R. T., et al., Remdesivir: a review of its discovery and development leading to emergency use authorization for treatment of COVID-19. ACS central Science, 2020. 6(5): p. 672-683.
[12] Cai, Q., et al., Experimental treatment with favipiravir for COVID-19: an open-label control study. Engineering, 2020. 6(10): p. 1192-1198.
[13] Good, S. S., et al., AT-527, a double prodrug of a guanosine nucleotide analog, is a potent inhibitor of SARS-CoV-2 in vitro and a promising oral antiviral for treatment of COVID-19. Antimicrobial Agents and Chemotherapy, 2021. 65(4): p. e02479-20.
[14] Pindiprolu, S. K. S. and S. H. Pindiprolu, Plausible mechanisms of Niclosamide as an antiviral agent against COVID-19. Medical hypotheses, 2020. 140: p. 109765.
[15] Horby, P. W., et al., Lopinavir-ritonavir in patients admitted to hospital with COVID-19 (RECOVERY): a randomised, controlled, open-label, platform trial. The Lancet, 2020. 396(10259): p. 1345-1352.
[16] Cox, R. M., J. D. Wolf, and R. K. Plemper, Therapeutically administered ribonucleoside analogue MK-4482/EIDD-2801 blocks SARS-CoV-2 transmission in ferrets. Nature microbiology, 2021. 6(1): p. 11-18.
[17] Imran, M., et al., Discovery, Development, and Patent Trends on Molnupiravir: A Prospective Oral Treatment for COVID-19. Molecules, 2021. 26(19): p. 5795.
[18] Ramos-Guzmán, C. A., J. J. Ruiz-Pernía, and I. Tuñón, Binding and Reactivity of a Nitrile Oral Inhibitor of SARS-CoV-2 Main Protease Revealed by Computational Simulations. 2021. 55
[19] Wen, W., et al., Efficacy and safety of three new oral antiviral treatment (molnupiravir, fluvoxamine and Paxlovid) for COVID-19: a meta-analysis. Annals of Medicine, 2022. 54(1): p. 516-523.
[20] Alzaabi, M. M., et al., Flavonoids are promising safe therapy against COVID-19. Phytochemistry Reviews, 2021: p. 1-22.
[21] Hamoda, A. M., et al., Marine sponge is a promising natural source of anti-SARS-CoV-2 scaffold.

Frontiers in pharmacology, 2021. 12.
[22] Chowdhary, M., et al., BRAF inhibitors and radiotherapy for melanoma brain metastases: potential advantages and disadvantages of combination therapy. OncoTargets and therapy, 2016. 9: p. 7149.
[23] 23. Gradman, A. H., et al., Combination therapy in hypertension. Journal of the American Society of Hypertension, 2010. 4(2): p. 90-98.
[24] Liu, Z., et al., A dual-inhibitor system for the effective antifibrillation of AB40 peptides by biodegradable EGCG-Fe (iii)/PVP nanoparticles. Journal of Materials Chemistry B, 2019. 7(8): p. 1292-1299.
[25] Patyar, S., A. Prakash, and B. Medhi, Dual inhibition: a novel promising pharmacological approach for different disease conditions. Journal of Pharmacy and Pharmacology, 2011. 63(4): p. 459-471.
[26] Chen, K., et al., Structure-Activity Relationships (SAR) Research of Thiourea Derivatives as Dual Inhibitors Targeting both HIV-1 Capsid and Human Cyclophilin A. Chemical biology & drug design, 2010. 76(1): p. 25-33.
[27] Wu, C., et al., A dual-functional molecular strategy for in situ suppressing and visualizing of neuraminidase in aqueous solution using iridium (iii) complexes. Chemical Communications, 2019. 55(45): p. 6353-6356.
[28] Cicconetti, F., et al., Extracellular pH, osmolarity, temperature and humidity could discourage SARS-CoV-2 cell docking and propagation via intercellular signaling pathways. PeerJ, 2021. 9: p. e12227.
[29] Mostafa, A., et al., FDA-approved drugs with potent in vitro antiviral activity against severe acute respiratory syndrome coronavirus 2. Pharmaceuticals, 2020. 13(12): p. 443.
[30] Elseginy, S. A., et al., Promising anti-SARS-CoV-2 drugs by effective dual targeting against the viral and host proteases. Bioorganic & Medicinal Chemistry Letters, 2021: p. 128099.
[31] Hoffmann, M., et al., SARS-CoV-2 cell entry depends on ACE2 and TMPRSS2 and is blocked by a clinically proven protease inhibitor. 2020. 181(2): p. 271-280. e8.
[32] Goc, A., et al., Simultaneous Inhibition of SARS-CoV-2 Infectivity by a Specific Combination of Plant-derived Compounds. European Journal of Biology and Biotechnology, 2021. 2(5): p. 24-33.
[33] Fu, L., et al., Both Boceprevir and GC376 efficaciously inhibit SARS-CoV-2 by targeting its main protease. 2020. 11(1): p. 1-8.
[34] Zhang, J., et al., Antiviral activity of tannin from the pericarp of Punica granatum L. against genital Herpes virus in vitro. Zhongguo Zhong yao za zhi=Zhongguo zhongyao zazhi=China journal of Chinese materia medica, 1995. 20(9): p. 556-8, 576, inside backcover.
[35] Kuo, Y.-C., et al., Samarangenin B from Limonium sinense suppresses herpes simplex virus type 1 replication in Vero cells by regulation of viral macromolecular synthesis. Antimicrobial agents and chemotherapy, 2002. 46(9): p. 2854-2864.
[36] Schuhmacher, A., J. Reichling, and P. Schnitzler, Virucidal effect of peppermint oil on the enveloped viruses herpes simplex virus type 1 and type 2 in vitro. Phytomedicine, 2003. 10(6-7): p. 504-510. 56
[37] Sastry, G. M., et al., Protein and ligand preparation: parameters, protocols, and influence on virtual screening enrichments. Journal of computer-aided molecular design, 2013. 27(3): p. 221-234.
[38] Harder, E., et al., OPLS3: a force field providing broad coverage of drug-like small molecules and proteins. Journal of chemical theory and computation, 2016. 12(1): p. 281-296.
[39] Giardina, S. F., et al., Novel, Self-Assembling Dimeric Inhibitors of Human B Tryptase. Journal of medicinal chemistry, 2020. 63(6): p. 3004-3027.
[40] Shelley, J. C., et al., Epik: a software program for pK a prediction and protonation state generation for drug-like molecules. Journal of computer-aided molecular design, 2007. 21(12): p. 681-691.
[41] Greenwood, J. R., et al., Towards the comprehensive, rapid, and accurate prediction of the favorable tautomeric states of drug-like molecules in aqueous solution. Journal of computer-aided molecular design, 2010. 24(6): p. 591-604.
[42] Ganai, S. A., et al., Combinatorial in silico strategy towards identifying potential hotspots during inhibition of structurally identical HDAC1 and HDAC2 enzymes for effective chemotherapy against neurological disorders. Frontiers in molecular neuroscience, 2017. 10: p. 357.
[43] Halgren, T. A., et al., Glide: a new approach for rapid, accurate docking and scoring. 2. Enrichment factors in database screening. Journal of medicinal chemistry, 2004. 47(7): p. 1750-1759.
[44] Friesner, R. A., et al., Glide: a new approach for rapid, accurate docking and scoring. 1. Method and assessment of docking accuracy. Journal of medicinal chemistry, 2004. 47(7): p. 1739-1749.
[45] Friesner, R. A., et al., Extra precision glide: Docking and scoring incorporating a model of hydrophobic enclosure for protein-ligand complexes. Journal of medicinal chemistry, 2006. 49(21): p. 6177-6196.
[46] 46. Daina, A., O. Michielin, and V. Zoete, SwissADME: a free web tool to evaluate pharmacokinetics, drug-likeness and medicinal chemistry friendliness of small molecules. Scientific reports, 2017. 7: p. 42717.
[47] 55. Chien, M., et al., Nucleotide analogues as inhibitors of SARS-CoV-2 polymerase, a key drug target for COVID-19. Journal of proteome research, 2020. 19(11): p. 4690-4697.
[48] Hempel, T., et al., Molecular mechanism of SARS-CoV-2 cell entry inhibition via TMPRSS2 by Camostat and Nafamostat mesylate. BioRxiv, 2020.
[49] Olomola, T. O., et al., Synthesis and evaluation of coumarin derivatives as potential dual-action HIV-1 protease and reverse transcriptase inhibitors. Bioorganic & medicinal chemistry, 2013. 21(7): p. 1964-1971.
[50] Patil, S. M., et al., In silico identification of novel benzophenone-coumarin derivatives as SARS-CoV-2 RNA-dependent RNA polymerase (RdRp) inhibitors. Journal of Biomolecular Structure and Dynamics, 2021: p. 1-17.
[51] Nair, A. B., Jacob S. (2016). A simple practical guide for dose conversion between animal and human. J Basic Clin Pharma 2016, 7:27-31.

1. A compound with dual inhibition activity against SARS-CoV-2 comprising (A) any of a coumarin or nucleoside analogue moiety selected from the group consisting of 2H-chromen-6-yl, adenosine, cytosine, pyrimidin, isocytosine, azacytosine, triazine, and (2R,3R,4S,5R)-2-(4-Amino-pyrrolo[2,1-f]-[1,2,4]-triazin-7-yl)-3,4-dihydroxy-5-hydroxymethyl-tetrahydro-furan-2-carbonitrile; (B) any of a 2-amino pyrimidine or guanidine analogue moiety selected form the group consisting of 4-guanidino-benzamide, 4-guanidino-benzoic acid, and [1,2,4]-triazin-4-yl; and (C) a linker selected from the group consisting of an amide, and an ester.

2. The compound of claim 1, comprising (A) a nucleoside analogue moiety; (B) a guanidine analogue moiety; and (C) an amide linker according to formula RH11 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-guanidino-benzamide) having the structure:

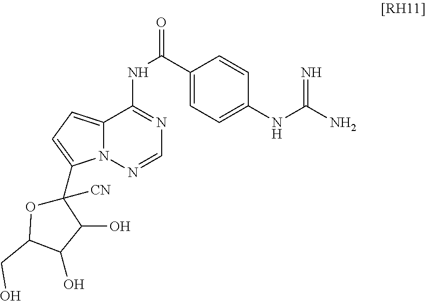 [SMILES: N#CC1(c2ccc3c(NC(=O)c4ccc(NC(=N)N)cc4)ncnn23)OC(CO)C(O)C1O]

3. The compound of claim 1, wherein the compound is according to formula RH12 (4-guanidino-benzoic acid 4-isopropyl-2-oxo-2H-chromen-6-yl ester) having the structure:

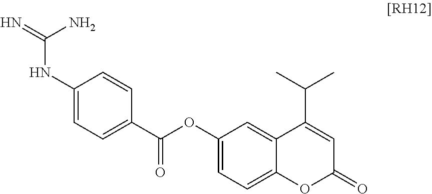 [SMILES: CC(C)c1cc(=O)oc2ccc(OC(=O)c3ccc(NC(=N)N)cc3)cc12]

4. The compound of claim 1, the compound being according to formula RH13 ((2R,3R,4S,5R)-N-[7-(2-cyano-3,4-dihydroxy-5-hydroxymethyl-tetrahydro-furan-2-yl)-pyrrolo[2,1-f]-[1,2,4]-triazin-4-yl]-4-(pyrimidin-2-ylamino)-benzamide) having the structure:

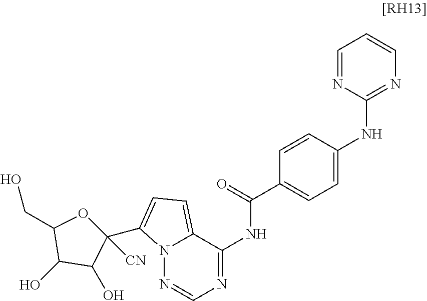 [SMILES: N#CC1(c2ccc3c(NC(=O)c4ccc(Nc5ncccn5)cc4)ncnn23)OC(CO)C(O)C1O]

5. The compound of claim 1, wherein the dual inhibitory activity of SARS-CoV-2 is targeted against the human TMPRSS2 enzyme and the SARS-CoV-2 RNA-dependent RNA polymerase enzyme.

6. The compound of claim 5, wherein the inhibitory activity of SARS-CoV-2 is additionally targeted against the SARS-CoV-2 M^{pro} enzyme.

7. A pharmaceutical composition, comprising a therapeutically effective amount of one or more of the compounds of claim 1, or pharmaceutically acceptable salts thereof, and one or more pharmaceutical excipients.

8. A method of treating a subject with SARS-CoV-2 infection, comprising administering to the subject in need thereof a therapeutically effective amount of the compound of claim 1, or pharmaceutically acceptable salt thereof, and one or more pharmaceutical excipients.

9. The method of claim 8, wherein the subject is a mammal.

10. The method of claim 9, wherein the mammal is a human.

11. The method of claim 10, wherein the subject has SARS-CoV-2 infection.

12. A kit for treating a subject with a SARS-CoV-2 infection, comprising the compound of claim 1, or a pharmaceutically acceptable salt thereof and one or more pharmaceutical excipients.
